(19) United States
(12) Patent Application Publication
(10) Pub. No.: US 2022/0162680 A1
(43) Pub. Date: May 26, 2022

(54) COMPOSITIONS AND METHODS FOR SEQUENCING USING FLUOROPHORES AND QUENCHERS OR DONORS

(71) Applicant: ILLUMINA, INC., San Diego, CA (US)

(72) Inventors: Jeffrey Mandell, Rancho Santa Fe, CA (US); Sergio Peisajovich, San Diego, CA (US); Seth McDonald, San Diego, CA (US); Kaitlin Pugliese, San Diego, CA (US)

(73) Assignee: ILLUMINA, INC., San Diego, CA (US)

(21) Appl. No.: 17/507,647

(22) Filed: Oct. 21, 2021

Related U.S. Application Data
(60) Provisional application No. 63/116,329, filed on Nov. 20, 2020.

(51) Int. Cl. C12Q 1/6818; C12Q 1/6834

(52) U.S. Cl. CPC C12Q 1/6818; C12Q 1/6834

(57) ABSTRACT

A method is provided that includes adding, using a polymerase coupled to a substrate, nucleotides in a solution to a first polynucleotide using at least a sequence of a second polynucleotide. The method includes separating, using labels respectively coupled to the nucleotides, quenchers from respective fluorophores. The method includes then detecting a sequence in which the polymerase adds the nucleotides to the first polynucleotide using at least fluorescence from the respective fluorophores.

### CROSS-REFERENCE TO RELATED APPLICATIONS

This application claims the benefit of U.S. Provisional Patent Application No. 63/116,329, filed Nov. 20, 2020 and entitled “Compositions and Methods for Sequencing Using Fluorophores and Quenchers or Donors,” the entire contents of which are incorporated by reference herein.

### BACKGROUND

The detection of specific nucleic acid sequences present in a biological sample has been used, for example, as a method for identifying and classifying microorganisms, diagnosing infectious diseases, detecting and characterizing genetic abnormalities, identifying genetic changes associated with cancer, studying genetic susceptibility to diseases, and measuring response to various types of treatment. A common technique for detecting specific nucleic acid sequences in a biological sample is nucleic acid sequencing.

Nucleic acid sequencing methodology has evolved from the chemical degradation methods used by Maxam and Gilbert and the strand elongation methods used by Sanger. Several sequencing methodologies are now in use which allow for the parallel processing of millions, or even billions, of nucleic acids on a single flow cell. Some platforms include bead-based and microarray formats in which silica beads are functionalized with probes depending on the application of such formats in applications including sequencing, genotyping, or gene expression profiling.

Some sequencing systems use fluorescence-based detection, whether for “sequencing-by-synthesis” or for genotyping, in which a given nucleotide is labeled with a fluorescent label, and the nucleotide is identified based on detecting the fluorescence from that label.

### SUMMARY

Examples provided herein are related to sequencing using fluorophore quenchers or donors. Methods for performing such sequencing, and associated compositions and devices, are disclosed.

In some examples, a method is provided herein. The method may include adding, using a polymerase coupled to a substrate, nucleotides in a solution to a first polynucleotide using at least a sequence of a second polynucleotide. The method may include separating, using labels respectively coupled to the nucleotides, quenchers from respective fluorophores. The method may include detecting a sequence in which the polymerase adds the nucleotides to the first polynucleotide using at least fluorescence from the respective fluorophores.

In some examples, the quenchers may be coupled to the substrate. In some examples, the fluorophores may be coupled to the substrate. In some examples, the fluorophores may be coupled to respective quenchers via non-covalent associations. In some examples, the separating may include the labels disrupting the non-covalent associations. In some examples, each of the fluorophores may be coupled to a respective first oligonucleotide, each of the quenchers may be coupled to a respective second oligonucleotide, and the non-covalent associations may include hybridizations between the first oligonucleotides and the second oligonucleotides. In some examples, each of the labels may include a respective third oligonucleotide. In some examples, the third oligonucleotides may disrupt the non-covalent associations by hybridizing to the first oligonucleotides. In some examples, the third oligonucleotides may disrupt the non-covalent associations by hybridizing to the second oligonucleotides.

In some examples, each of the fluorophores and respective quencher may be coupled to a respective hairpin oligonucleotide having first and second stem sequences and a loop sequence, and the non-covalent associations may include hybridizations between the first and second stem sequences. In some examples, each of the labels may include a respective oligonucleotide. In some examples, the oligonucleotides may disrupt the non-covalent associations by hybridizing to the loop sequences. In some examples, after the polymerase adds respective nucleotides, the non-covalent associations may re-form between the fluorophores and respective quenchers.

In some examples, the quenchers may be in the solution. In some examples, the fluorophores may be coupled to respective labels. In some examples, the fluorophores may be coupled to respective quenchers via non-covalent associations. In some examples, an element coupled to the surface may disrupt the non-covalent associations. In some examples, each of the labels may be coupled to a respective first oligonucleotide to which a respective fluorophore is coupled, each of the quenchers may be coupled to a respective second oligonucleotide, and the non-covalent associations may include hybridizations between the first oligonucleotides and the second oligonucleotides. In some examples, the element coupled to the surface may include a third oligonucleotide. In some examples, the third oligonucleotide may disrupt the non-covalent associations by hybridizing to the first oligonucleotide. In some examples, the third oligonucleotide may disrupt the non-covalent associations by hybridizing to the second oligonucleotides.

In some examples, each of the fluorophores and respective quencher may be coupled to a respective hairpin oligonucleotide having first and second stem sequences and a loop sequence, and the non-covalent associations may include hybridizations between the first and second stem sequences. In some examples, the element coupled to the surface may include an oligonucleotide. In some examples, the oligonucleotide may disrupt the non-covalent associations by hybridizing to the loop sequence.

In some examples, after the polymerase adds respective nucleotides, new non-covalent associations may form between the fluorophores and respective quenchers in the solution.

In some examples, a composition is provided herein. The composition may include a substrate; a polymerase coupled to the substrate; a plurality of quenchers coupled to the substrate; and a plurality of fluorophores. Each fluorophore may be non-covalently associated with at least one of the quenchers.

In some examples, the composition further may include a solution including nucleotides having labels coupled thereto, and optical detection circuitry. The polymerase may be to add the nucleotides to a first polynucleotide using at least a sequence of a second polynucleotide. The labels may disrupt the non-covalent associations between the fluorophores and the quenchers. The optical detection circuitry may be to detect fluorescence from the fluorophores resulting from the disruption in the non-covalent associations.

In some examples, the fluorophores may be are coupled to the substrate. In some examples, each of the fluorophores may be coupled to a respective first oligonucleotide, each of the quenchers may be coupled to a respective second oligonucleotide, and the non-covalent associations may include hybridizations between the first oligonucleotides and the second oligonucleotides. In some examples, each of the labels may include a respective third oligonucleotide. In some examples, the third oligonucleotide may disrupt the non-covalent associations by hybridizing to the first oligonucleotide. In some examples, the third oligonucleotide may disrupt the non-covalent associations by hybridizing to the second oligonucleotide.

In some examples, each of the fluorophores and respective quencher may be coupled to a respective hairpin oligonucleotide having first and second stem sequences and a loop sequence, and the non-covalent associations may include hybridizations between the first and second stem sequences. In some examples, each of the labels may include a respective oligonucleotide. In some examples, the oligonucleotides may disrupt the non-covalent associations by hybridizing to the loop sequences. In some examples, after the polymerase adds respective nucleotides, the non-covalent associations may re-form between the fluorophores and respective quenchers.

In some examples, a composition is provided herein. The composition may include a substrate; a polymerase coupled to the substrate; and a solution including (i) quenchers and (ii) nucleotides coupled to labels. Each label may be coupled to a fluorophore. Each fluorophore may be non-covalently associated with at least one of the quenchers. The composition may include an element coupled to the substrate to disrupt the non-covalent associations between the fluorophores and the quenchers.

In some examples, the composition further may include optical detection circuitry. The polymerase may be to add the nucleotides to a first polynucleotide using at least a sequence of a second polynucleotide. The optical detection circuitry may be to detect fluorescence from the fluorophores resulting from the disruption in the non-covalent associations. In some examples, each of the labels may be coupled to a respective first oligonucleotide to which a respective fluorophore is coupled, each of the quenchers may be coupled to a respective second oligonucleotide, and the non-covalent associations may include hybridizations between the first oligonucleotides and the second oligonucleotides. In some examples, the element coupled to the surface may include a third oligonucleotide. In some examples, the third oligonucleotide may be to disrupt the non-covalent associations by hybridizing to the first oligonucleotides. In some examples, the third oligonucleotide may be to disrupt the non-covalent associations by hybridizing to the second oligonucleotides.

In some examples, each of the fluorophores and respective quencher may be coupled to a respective hairpin oligonucleotide having first and second stem sequences and a loop sequence, and the non-covalent associations may include hybridizations between the first and second stem sequences. In some examples, the element coupled to the surface may include an oligonucleotide. In some examples, the oligonucleotide may be to disrupt the non-covalent associations by hybridizing to the loop sequences.

In some examples, after the polymerase adds respective nucleotides, new non-covalent associations may form between the fluorophores and respective quenchers in the solution.

In some examples, a method is provided herein. The method may include adding, using a polymerase coupled to a substrate, nucleotides in a solution to a first polynucleotide using at least a sequence of a second polynucleotide. The method may include exciting, using energy transfer from a donor coupled to the substrate, fluorophores coupled to respective nucleotides. The method may include detecting a sequence in which the polymerase adds the nucleotides to the first polynucleotide using at least fluorescence from the respective excited fluorophores.

In some examples, exciting the fluorophores may include optically exciting the donor at a first time. In some examples, optically exciting the donor at the first time also may optically excite the fluorophores coupled to the respective nucleotides. In some examples, the fluorophores may have a first emission lifetime after the optical excitation, and the donor may have a second emission lifetime after the optical excitation that is longer than the first emission lifetime. In some examples, the sequence may be detected using fluorescence from the respective excited fluorophores beginning at a second time after the first lifetime ends.

In some examples, each of the fluorophores may be coupled to a respective first oligonucleotide coupled to a respective nucleotide, the donor may be coupled to a respective second oligonucleotide coupled to the substrate, each of the first oligonucleotides may hybridize to the second oligonucleotide while the polymerase adds that nucleotide to the first polynucleotide, and the energy may transfer from the donor to that fluorophore while the first oligonucleotide is hybridized to the second oligonucleotide.

In some examples, the donor may include a rare earth element. In some examples, the donor may include a nanoparticle. In some examples, the donor may include a rare earth element chelate or cryptate. In some examples, the fluorophore may include a quantum dot.

In some examples, the donor may include a plurality of donors each coupled to the substrate. In some examples, each of the fluorophores may be coupled to a respective first oligonucleotide coupled to a respective nucleotide, the donor may include a plurality of donors each coupled to a respective second oligonucleotide coupled to the substrate, each of the first oligonucleotides may hybridize to a corresponding one of the second oligonucleotides while the polymerase adds that nucleotide to the first polynucleotide, and the energy may transfer from the respective donor to that fluorophore while the first oligonucleotide is hybridized to the second oligonucleotide.

In some examples, a composition is provided herein. The composition may include a substrate; a polymerase coupled to the substrate; a donor coupled to the substrate; and a solution including nucleotides coupled to respective fluorophores.

In some examples, the composition further may include optical detection circuitry. The polymerase may be to add the nucleotides to a first polynucleotide using at least a sequence of a second polynucleotide, and the optical detection circuitry may be to detect fluorescence from the fluorophores resulting from energy transfer from the donor to fluorophores coupled to respective nucleotides.

In some examples, the composition further may include an excitation light source to optically excite the donor at a first time, the optically excited donor exciting the fluorophores using the energy transfer. In some examples, optically exciting the donors at the first time also may optically excite the fluorophores coupled to the respective nucleotides. In some examples, the fluorophores may have a first emission lifetime after the optical excitation, and the donor may have a second emission lifetime after the optical excitation that is longer than the first emission lifetime. In some examples, the sequence may be detected using fluorescence from the respective excited fluorophores beginning at a second time after the first emission lifetime ends.

In some examples, each of the fluorophores may be coupled to a respective first oligonucleotide coupled to a respective nucleotide, the donor may be coupled to a respective second oligonucleotide coupled to the substrate, each of the first oligonucleotides may hybridize to the second oligonucleotide while the polymerase adds that nucleotide to the first polynucleotide, and the energy may transfer from the donor to that fluorophore while the first oligonucleotide is hybridized to the second oligonucleotide.

In some examples, the donor may include a rare earth element. In some examples, the donor may include a nanoparticle. In some examples, the donor may include a rare earth element chelate or cryptate. In some examples, the fluorophore may include a quantum dot.

In some examples, the donor may include a plurality of donors each coupled to the substrate. In some examples, each of the fluorophores may be coupled to a respective first oligonucleotide coupled to a respective nucleotide, the donor may include a plurality of donors each coupled to a respective second oligonucleotide coupled to the substrate, each of the first oligonucleotides may hybridize to a corresponding one of the second oligonucleotides while the polymerase adds that nucleotide to the first polynucleotide, and the energy may transfer from the respective donor to that fluorophore while the first oligonucleotide is hybridized to the second oligonucleotide.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate example compositions and operations in a process flow for sequencing using fluorophores and quenchers.

FIGS. 2A-2B schematically illustrate example compositions and operations in a process flow for sequencing using fluorophores and quenchers.

FIGS. 3A-3B schematically illustrate example compositions and operations in a process flow for sequencing using fluorophores and quenchers.

FIGS. 4A-4B schematically illustrate example compositions and operations in a process flow for sequencing using fluorophores and quenchers.

FIGS. 5A-5B schematically illustrate example compositions and operations in a process flow for sequencing using fluorophores and quenchers.

FIGS. 6A-6D schematically illustrate example compositions and operations in a process flow for sequencing using fluorophores and donors.

FIGS. 7A-7C schematically illustrate example emission lifetimes in a process flow for sequencing using fluorophores and donors.

FIGS. 8A-8B schematically illustrates example compositions and operations in a process flow for sequencing using fluorophores and quenchers.

FIG. 9 illustrates an example flow of operations in a method for sequencing using fluorophores and quenchers.

FIG. 10 illustrates an example flow of operations in a method for sequencing using fluorophores and donors.

FIG. 11 illustrates an example labeled nucleotide that may be used in the present compositions and methods.

FIGS. 12A-12B schematically illustrate plots of example fluorescence measurements.

FIGS. 13A-13B schematically illustrate example compositions and operations in a process flow for sequencing using fluorophores and quenchers.

### DETAILED DESCRIPTION

Sequencing polynucleotides using fluorophores and quenchers or donors is provided herein.

For example, the present application relates to fluorescence-based, single-molecule, real-time sequencing technologies. Previously known fluorescence-based, single-molecule sequencing technologies may use zero-mode waveguides (ZMWs) to eliminate signal contributions that otherwise may arise from background fluorescence caused by fluorophores in solution. For example, a polymerase located in the well of a ZMW may sequentially add nucleotides labeled with fluorophores to a growing polynucleotide, using the sequence of a polynucleotide being sequenced as a template. The ZMW may optically limit excitation light from the light source to the polymerase and incorporating nucleotides, thus facilitating detection of fluorescence from incorporating nucleotides over those that are in solution. However, because ZMWs are relatively small and may be relatively deep, the polymerase may preferentially capture polynucleotides that are relatively small, leading to a sequencing bias.

In comparison, provided herein are methods and compositions for fluorescence-based, single-molecule, real-time sequencing that need not necessarily be used with ZMWs, although the present compositions and methods suitably may be adapted for use with ZMWs. More specifically, instead of relying upon the optical limiting of excitation light, the present methods and compositions provide for sequencing without such limitation of excitation light, and with sufficiently low background fluorescence, e.g., substantially no background fluorescence, that signals from fluorophores readily may be detected on a single-molecule basis as a polymerase adds nucleotides to a growing polynucleotide using the sequence of a polynucleotide being sequenced as a template. In some examples, interaction between the polymerase and a labeled nucleotide causes dissociation between a fluorophore and a quencher, resulting in fluorescence that can be used to identify the nucleotide. In other examples, interaction between the polymerase and a labeled nucleotide causes association between a fluorophore and a donor, resulting in fluorescence that can be used to identify the nucleotide.

Some terms used herein will be briefly explained. Then, some example systems and example methods for sequencing polynucleotides using quenchers or donors, and associated compositions and devices, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” refers to a polynucleotide to which nucleotides may be added via a free 3′ OH group. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. A primer may be coupled to a substrate.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, Calif.). Another example patterned substrate having these characteristics is a zero-mode waveguide (ZMW).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, Calif.).

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “fluorophore” is intended to mean an element that emits light at a first wavelength (“emission,” or “fluorescence”) responsive to excitation with light at a second wavelength (“optical excitation,” or “excitation light”) that is different from the first wavelength. The light emitted by a fluorophore may be referred to as “fluorescence” and may be detected by suitable optical circuitry. The light emitted by a fluorophore may have an “emission lifetime” that characterizes the intensity as a function of time with which the fluorophore fluoresces after optical excitation. In various examples, a fluorophore may be or include a molecule such as an organic dye, a fluorescent protein, or a particle such as a quantum dot. Example organic dyes include xanthene derivatives (such as fluorescein and rhodamine and their derivatives), cyanine and its derivatives, squaraine derivatives and ring-substituted squaraines, squaraine rotaxane derivatives, naphthalene derivatives, coumarin derivatives, oxadiazole derivatives, anthracene derivatives, pyrene derivatives, oxazine derivatives, acridine derivatives, arylmethine derivatives, tetrapyrrole derivatives, and dipyrromethene derivatives. Example fluorescent proteins include green fluorescent protein (GFP), yellow fluorescent protein (YFP), and red fluorescent protein (RFP). Some specific, nonlimiting examples of organic dyes that may be used as fluorophores include rhodamine and its derivatives such as TMR (tetramethylrhodamine), TAMRA (carboxytetramethylrhodamine), or 5TAMRA (5-carboxytetramethylrhodamine); fluorescein and its derivatives such as FAM (fluorescein amidite) or 5FAM (5-carboxyfluorescein); cyanine and its derivatives such as cyanine3 (Cy3, 1-[6-(6-aminohexylamino)-6-oxohexyl]-3,3-dimethyl-2-[(1E,3E)-3-(1,3,3-trimethylindolin-2-ylidene)prop-1-enyl]-3H-indolium chloride hydrochloride) or cyanine5 (Cy5, 1-[6-(2,5-dioxopyrrolidin-1-yloxy)-6-oxohexyl]-3,3-dimethyl-2-[(1E,3E,5E)-5-(1,3,3-trimethylindolin-2-ylidene)penta-1,3-dienyl]-3H-indolium tetrafluoroborate); BODIPY (4,4-difluoro-4-bora-3a,4a-diaza-s-indacene); Atto dyes commercially available from Millipore Sigma; XL665 commercially available from CisBio (phycobilliprotein pigment purified from red algae); or Alexa Fluor dyes commercially available from ThermoFisher Scientific, such as Alexa Fluor 680, Alexa Fluor 488, Alexa Fluor 546, Alexa Fluor 633, and Alexa Fluor 647. In still other examples, the fluorophore may include a quantum dot.

By “quantum dot” it is meant a particle including about 100 to about 100,000 atoms and a diameter of about 2 to about 10 nm, and that emits light in response to excitation light or energy transfer. Quantum dots may include, or may consist essentially of, inorganic atoms. Quantum dots may include atoms from groups II-IV, groups III-V, or groups IV-VI of the period table, and may include a core having a first composition that is covered by a shell having a second, different composition. Cadmium (Cd) may be included in the core and/or in the shell. In one nonlimiting example, a quantum dot includes a CdSe core covered by a ZnS shell, and may be referred to as a CdSe/ZnS core/shell quantum dot. In another nonlimiting example, a quantum dot includes a CdSe core covered by a CdS shell, and may be referred to as a CdSe/ZnS core/shell quantum dot. Quantum dots may have relatively narrow emission peaks, and may have a brighter emission and a higher signal to noise ratio as compared to organic dyes (e.g., may be about 10-20 times brighter than organic dyes). Quantum dots also may be relatively stable because their inorganic composition may inhibit the effect of photobleaching. Quantum dots also may have a significantly longer fluorescence time (e.g., about 10-40 ns) as compared to that of inorganic dyes (e.g., a few nanoseconds).

As used herein, the term “quencher” is intended to mean an element that, when in sufficient proximity to a fluorophore, reduces or inhibits fluorescence from that fluorophore. In various examples, a quencher may be or include a molecule. Example commercially available quenchers include DABCYL (dimethylaminoazobenzenesulfonic acid, Jena Bioscience GMBH), Black Hole Quencher dyes (BHQ, LGC Biosearch Technologies), IOWA BLACK® FQ (Integrated DNA Technologies), IOWA BLACK® RQ (Integrated DNA Technologies), and IRDYE® QC-1 (LI-COR Biosciences).

As used herein, the term “donor” is intended to mean an element that, when optically excited in sufficient proximity to an “acceptor”, transfers energy to the acceptor. As used herein, the term “acceptor” is intended to mean an element that receives transferred energy from a donor, and fluoresces responsive to such energy transfer. The energy transferred from the donor may be at a suitable wavelength to optically excite the acceptor. In some examples, the acceptor may be or include a fluorophore. For example, an acceptor may fluoresce both in response to excitation light, and in response to energy transfer from a donor. Similarly, in some examples, the donor may be or include a fluorophore. For example, a donor may both fluoresce in response to excitation light, and may transfer energy to an acceptor. However, a donor may transfer energy to an acceptor through a mechanism other than fluorescence, e.g., may transfer energy to an acceptor via luminescence. In some examples, the mechanism via which energy is transferred from a donor to an acceptor may be or include Förster resonance energy transfer (FRET). A donor and acceptor may be coupled to the same element as one another in some examples, and together may be considered to provide a “fluorophore” as the term is used herein.

In some examples, a donor may include a rare earth element, which also may be referred to as a lanthanide. Example rare earth elements that may be used as a donor include lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). Specific examples of lanthanide donors include Sm(III), Eu(III), Tb(III), and Dy(III). The donor (e.g., rare earth element) may be provided in any suitable form. For example, the donor may include a nanoparticle (illustratively, having a diameter of about 1 nm to about 30 nm, or about 5 nm to about 20 nm). Or, for example, the donor may include a rare earth element chelate or cryptate. Donors and acceptors may be used as “pairs” in which the donor and acceptor are selected such that the donor transfers energy at a suitable wavelength to optically excite the acceptor. Non-limiting examples of donor-acceptor pairs, which may be referred to as FRET pairs, include: terbium (donor) and TAMRA, BODIPY, GFP, Alexa Fluor 488, YFP, rhodamine, Alexa Fluor 546, Alexa Fluor 633, or Alexa Fluor 647, FAM, 5FAM, 5TAMRA, TMR or fluorescein (acceptors); europium chelate (donor) and Alexa Fluor 647 or Cy5 (acceptors); Cy3 (donor) and Cy5 (acceptor); Eu (III)-chelate nanoparticle (donor) and quantum dot, Alexa Fluor680 (acceptor); cryptate (donor) and XL665 (acceptor); and terbium or europium (donor) and a quantum dot, such as a CdSe/ZnS core/shell quantum dot (acceptor).

As used herein, to “detect” fluorescence is intended to mean to distinguish the emission of light from a fluorophore over that of background. Detection may include the generation of an electrical signal based on the received light, and the determination, using the electrical signal, that light was received from the fluorophore. Fluorescence may be detected using any suitable optical detection circuitry, which may include an optical detector to generate an electrical signal based on the light received from the fluorophore, and electronic circuitry to determine, using the electrical signal, that light was received from the fluorophore. As one example, the optical detector may include an active-pixel sensor (APS) including an array of amplified photodetectors configured to generate an electrical signal based on light received by the photodetectors. APSs may be based on complementary metal oxide semiconductor (CMOS) technology known in the art. CMOS-based detectors may include field effect transistors (FETs), e.g., metal oxide semiconductor field effect transistors (MOSFETs). In particular examples, a CMOS imager having a single-photon avalanche diode (CMOS-SPAD) may be used, for example, to perform fluorescence lifetime imaging (FLIM). In other examples, the optical detector may include a photodiode, such as an avalanche photodiode, charge-coupled device (CCD), cryogenic photon detector, reverse-biased light emitting diode (LED), photoresistor, phototransistor, photovoltaic cell, photomultiplier tube (PMT), optical microscope, near-field imager where the excitation source and optical detector are separated by less than a few wavelengths of light as in total internal reflection microscopy, quantum dot photoconductor or photodiode, or the like. The optical detection circuitry further may include any suitable combination of hardware and software in operable communication with the optical detector so as to receive the electrical signal therefrom, and configured to detect the fluorescence based on such signal, e.g., based on the optical detector detecting light from the fluorophore. For example, the electronic circuitry may include a memory and a processor coupled to the memory. The memory may store instructions for causing the processor to receive the signal from the optical detector and to detect the fluorophore using such signal. For example, the instructions can cause the processor to determine, using the signal from the optical detector, that fluorescence is emitted within the field of view of the optical detector and to determine, using such determination, that a fluorophore has fluoresced.

To “measure” fluorescence is intended to mean to determine a relative or absolute amount of the fluorescence that is detected. For example, the amount of fluorescence may change as a function of time, and changes in the amount of fluorescence may be measured relative to the initial amount of fluorescence, or as an absolute amount of fluorescence. Illustratively, the amount of fluorescence from a given fluorophore may vary according to its emission lifetime following excitation, and whether a donor transfers energy to it in which case fluorescence may persist after its emission lifetime otherwise would have ended. The memory of the electronic circuitry described above may store instructions causing the processor to monitor the level of the electrical signal at one or more times, and to correlate such level(s) to times during or after the emission lifetime of the fluorophore. For example, the electronic circuitry may store instructions to measure the time-resolved fluorescence (TRF) from the fluorophore.

As used herein, “non-covalent” association includes hydrogen bonds, ionic bonds, dipole-dipole bonds, London dispersion forces, or any suitable combination thereof. For example, complementary polynucleotides may non-covalently associate with one another. Protein-protein interactions and peptide-peptide interactions are other nonlimiting examples of non-covalent associations, including but not limited to coiled coil, leucine zipper, barnase-barstar, Ras-Raf, and general ligand-receptor associations.

Example Methods for Sequencing Using Fluorophores and Quenchers, and Associated Compositions

In some examples provided herein, polynucleotides may be sequenced on a single-molecule basis using fluorophores and quenchers. For example, a polymerase coupled to a substrate may add nucleotides in a solution to a first polynucleotide using at least a sequence of a second (“target”) polynucleotide as a template. Labels respectively coupled to the nucleotides may cause quenchers to separate from respective fluorophores. A sequence in which the polymerase adds the nucleotides to the first polynucleotide may be detected using at least fluorescence from the respective fluorophores. As described with regards to various examples below, any suitable ones of the quenchers, fluorophores, and other elements may be coupled to the substrate, may be in solution, or may be coupled to the nucleotides.

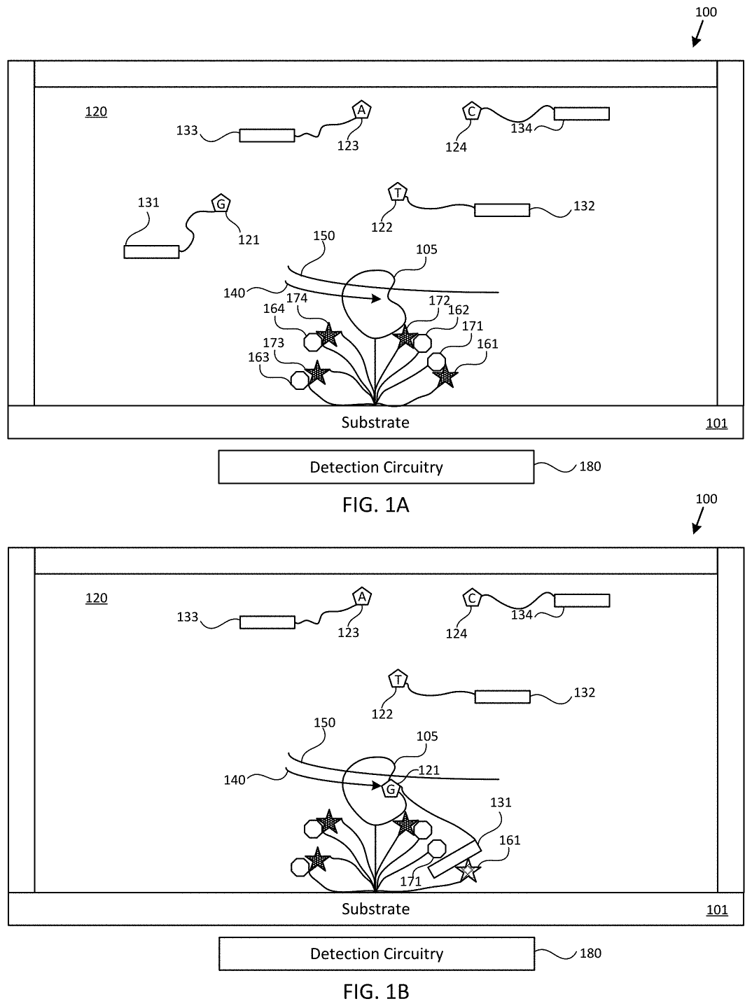

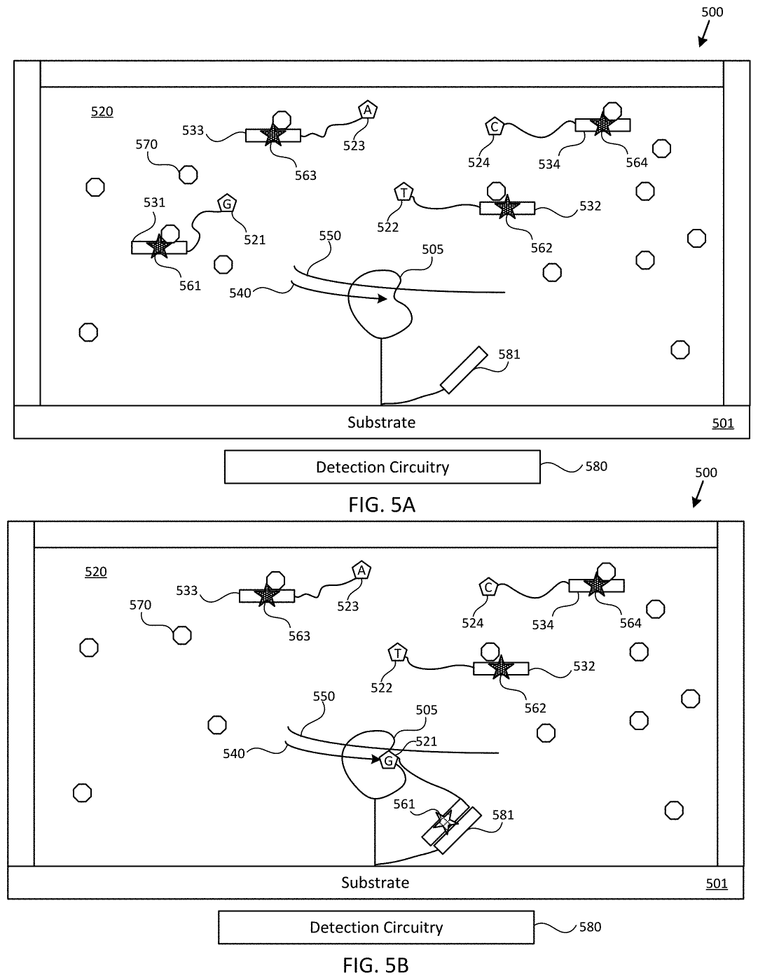

For example, FIGS. 1A-1B schematically illustrate example compositions and operations in a process flow for sequencing using fluorophores and quenchers. Composition 100 illustrated in FIG. 1A includes substrate 101, polymerase 105 coupled to the substrate, a plurality of quenchers 171, 172, 173, 174, a plurality of fluorophores 161, 162, 163, 164, each fluorophore being non-covalently associated with at least one of the quenchers. Illustratively, fluorophore 161 is non-covalently associated with quencher 171, fluorophore 162 is non-covalently associated with quencher 172, fluorophore 163 is non-covalently associated with quencher 173, and fluorophore 164 is non-covalently associated with quencher 174. At the particular time illustrated in FIG. 1A, quencher 171 may inhibit fluorescence from fluorophore 161, quencher 172 may inhibit fluorescence from fluorophore 162, quencher 173 may inhibit fluorescence from fluorophore 163, and quencher 174 may inhibit fluorescence from fluorophore 164, as suggested by the darkened stars in FIG. 1A. In the nonlimiting example illustrated in FIG. 1A, fluorophores 161, 162, 163, 164 are coupled to substrate 101 via respective linkers, while in other examples such as described with reference to FIGS. 5A-5B, the fluorophores instead may be in solution. In the nonlimiting example illustrated in FIG. 1A, quenchers 171, 172, 173, 174 are coupled to substrate 101 via respective linkers, while in other examples such as described with reference to FIGS. 5A-5B, the quenchers instead may be in solution.

Composition 100 illustrated in FIG. 1A includes a solution including nucleotides 121, 122, 123, 124 respectively having labels 131, 132, 133, 134 coupled thereto, as well as optical detection circuitry 180. Polymerase 105 is to add the nucleotides to first polynucleotide 140 using at least a sequence of second polynucleotide 150. Labels 131, 132, 133, 134 disrupt the non-covalent associations between the fluorophores and the quenchers, and optical detection circuitry 180 is to detect fluorescence from the fluorophores resulting from the disruption in the non-covalent associations. At the particular time illustrated in FIG. 1B, polymerase 105 is acting upon nucleotide 121 (illustratively, G) which maintains label 131 at a location that is sufficiently close to separate fluorophore 161 from quencher 171. Accordingly, fluorophore 161 may fluoresce in response to suitable optical excitation as suggested in FIG. 1B as illustrated by the lightened star, and optical detection circuitry 180 may detect such fluorescence.

In some examples, labels 131, 132, 133, 134 may selectively separate a particular fluorophore from a particular quencher. For example, at another time (not specifically illustrated), polymerase 105 may act upon nucleotide 122 (illustratively, T) which maintains label 132 at a location that is sufficiently close to selectively separate fluorophore 162 from quencher 172, such that fluorophore 162 may fluoresce in response to suitable optical excitation. At another time (not specifically illustrated), polymerase 105 may act upon nucleotide 123 (illustratively, A) which maintains label 133 at a location that is sufficiently close to selectively separate fluorophore 1632 from quencher 173, such that fluorophore 162 may fluoresce in response to suitable optical excitation. At another time (not specifically illustrated), polymerase 105 may act upon nucleotide 124 (illustratively, C) which maintains label 134 at a location that is sufficiently close to selectively separate fluorophore 164 from quencher 174, such that fluorophore 164 may fluoresce in response to suitable optical excitation. Fluorophores 161, 162, 163, 164 may emit fluorescence of different wavelengths than one another, e.g., may be different types of fluorophores than each other, so that nucleotides 121, 122, 123, 124 may be distinguished from one another using the respective fluorescence from those fluorophores (e.g., as detected using optical detection circuitry 180). For example, nucleotide 121 may be identified using fluorescence from fluorophore 161, nucleotide 122 may be identified using fluorescence from fluorophore 162, nucleotide 123 may be identified using fluorescence from fluorophore 163, and nucleotide 124 may be identified using fluorescence from fluorophore 164. Quenchers 171, 172, 173, 174 suitably may be selected so as to selectively and non-covalently associate with, fluorophores 161, 162, 163, 164, and to respectively inhibit fluorescence therefrom. Quenchers 171, 172, 173, 174 may be, but need not necessarily be, different types of quencher than one another.

So as to inhibit photobleaching of donors and acceptors 161, 162, 163, 164 over the course of sequencing polynucleotide 150, such agents may be selected so as to include highly stable organic dyes, or donor-acceptor pairs. Example highly stable organic dyes include Atto dyes (commercially available from Millipore Sigma), and Alexa Fluor dyes (commercially available from ThermoFisher Scientific). Example donors are provided elsewhere herein, and may include trivalent lanthanides such as Sm(III), Eu(III), Tb(III), and Dy(III), and chelates or cryptates thereof. Example acceptors that may be used with lanthanide donors are provided elsewhere herein, and may include but are not limited to quantum dots. The donor (e.g., lanthanide) and acceptor (e.g., quantum dot) both may be coupled to the same element as one another, and together may be considered to provide a “fluorophore” as the term is used herein. Note that both lanthanides (example donors) and quantum dots (example acceptors) may inhibit photobleaching, and thus may remain relatively stable over the course of many measurements. Additionally, or alternatively, solution 120 may include an antioxidant that inhibits photobleaching. Example antioxidants include reactive oxide species such as n-propyl gallate and ascorbic acid, oxygen scavenging systems such as glucose oxidase and catalase, and the use of micron-sized PDMS (polydimethylsiloxane) substrate wells treated with oxygen plasma and coated with 2-[methoxy(polyethyleneoxy)propyl]trimethoxysilane (PEG) (Gelest, Morrisville, Pa.).

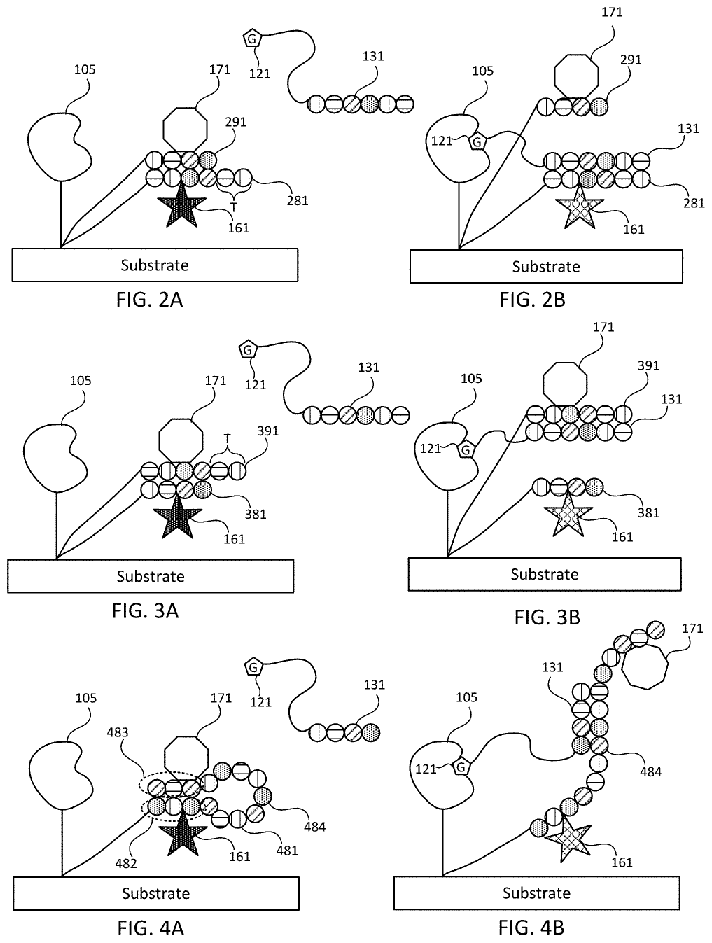

Any suitable non-covalent association between fluorophores 161, 162, 163, 164 and respective quenchers 171, 172, 173, 174 may be used. For example, each of the fluorophores may be coupled to a respective first oligonucleotide, each of the quenchers may be coupled to a respective second oligonucleotide, and the non-covalent associations comprise hybridizations between the first oligonucleotides and the second oligonucleotides. For example, FIGS. 2A-2B schematically illustrate example compositions and operations in a process flow for sequencing using fluorophores and quenchers. In the simplified example shown in FIG. 2A (in which fluorophores 162, 163, 164 and quenchers 172, 173, 174 are omitted for clarity), fluorophore 161 is coupled to oligonucleotide 281 which is coupled via a linker to the substrate, and quencher 171 is coupled to oligonucleotide 291 which is coupled via a linker to the substrate. Oligonucleotide 291 is hybridized to oligonucleotide 281 which places quencher 171 sufficiently close to inhibit fluorescence of fluorophore 161 as suggested by the darkened star. Each of the labels may include a respective third oligonucleotide. In the simplified example shown in FIG. 2A (in which nucleotides 122, 123, 124 and labels 132, 133, 134 are omitted for clarity), label 131 of nucleotide 121 includes an oligonucleotide having a sequence that is complementary to oligonucleotide 281. For example, as illustrated in FIG. 2B, the oligonucleotide of label 131 may disrupt the non-covalent association between oligonucleotides 281, 291 by hybridizing to oligonucleotide 281. In one example, the oligonucleotide label of 131 disrupts the association of oligonucleotides 281, 291 because the interaction between oligonucleotides 131 and 281 is more stable than that of oligonucleotides 281 and 291.

In some examples, to increase the kinetic association between oligonucleotide 131 and oligonucleotide 281 and the displacement of oligonucleotide 291, oligonucleotide 281 may include a toehold overhang T that provides a single-stranded region for oligonucleotide 131 to initiate the displacement of oligonucleotide 291. Additionally, or alternatively, to increase the kinetic association between oligonucleotides 131 and 281 and the displacement of oligonucleotide 291, the sequences of oligonucleotides 281 and 291 may have an internal mismatch whereas the sequences of oligonucleotides 131 and 281 may match fully. Additionally, or alternatively, oligonucleotides 131, 281, and 291 may have the same length as one another, or may have different lengths than one another. Additionally, or alternatively, oligonucleotide 131 may be or include a different type of oligonucleotide than does oligonucleotide 291, and is selected so as to interact more strongly with oligonucleotide 281 than does oligonucleotide 291. For example, oligonucleotides 281 and 291 may include DNA, and oligonucleotide 131 may include PNA or LNA that interacts more strongly with oligonucleotide 281 than does oligonucleotide 291 and therefore displaces oligonucleotide 291. Additionally, or alternatively, intercalators (such as quinolone) may interact with specific sequences in the DNA, e.g., through pi-pi stacking and hydrophobic interactions, or may form a complex with metal, and thus may be used to alter the Tm of the duplex between oligonucleotide 131 and oligonucleotide 281 and/or the Tm of the duplex between oligonucleotide 281 and oligonucleotide 291. In any such examples, or in other examples, the duplex between oligonucleotide 131 and oligonucleotide 281 may have a higher Tm than does the duplex between oligonucleotide 281 and oligonucleotide 291. The relative Tms of the duplex between oligonucleotide 131 and oligonucleotide 281 and the duplex between oligonucleotide 291 and oligonucleotide 281 may be governed, at least in part, by the relative kinetics of association between such pairs of oligonucleotides and may be adjusted, for example, by suitably adjusting relative on-rates, binding constants, and off-rates, all of which may vary with concentration (which is achieved locally when the nucleotide binds the polymerase active site).

As a result of such disruption between oligonucleotides 281 and 291, fluorophore 161 may be sufficiently separated from quencher 171 as to fluoresce, as suggested in FIG. 2B by the lightened star. After polymerase 105 finishes adding nucleotide 121 to a growing polynucleotide (not specifically illustrated), then label 131 may be cleaved and may dissociate from oligonucleotide 281, leaving oligonucleotide 281 available to rehybridize to oligonucleotide 291. As a result of such rehybridizing, fluorophore 161 may be sufficiently close to quencher 171 as substantially not to fluoresce. Note that in some examples (not specifically illustrated), oligonucleotide 291 need not necessarily be coupled to the substrate, and as such may dissociate into solution, and subsequently rehybridize out of solution. In such examples, oligonucleotide 291 in solution may interact more strongly with oligonucleotide 281 than does a label 131 that has been cleaved from nucleotide 121.

FIGS. 3A-3B schematically illustrate example compositions and operations in another process flow for sequencing using fluorophores and quenchers. In the simplified example shown in FIG. 3A (in which fluorophores 162, 163, 164 and quenchers 172, 173, 174 are omitted for clarity), fluorophore 161 is coupled to oligonucleotide 381 which is coupled via a linker to the substrate, and quencher 171 is coupled to oligonucleotide 391 which is coupled via a linker to the substrate. Oligonucleotide 391 is hybridized to oligonucleotide 381 which places quencher 171 sufficiently close to inhibit fluorescence of fluorophore 161 as suggested by the darkened star, similarly as described with reference to FIG. 2A. Each of the labels may include a respective third oligonucleotide. In the simplified example shown in FIG. 3A (in which nucleotides 122, 123, 124 and labels 132, 133, 134 are omitted for clarity), label 131 of nucleotide 121 includes an oligonucleotide having a sequence that is complementary to oligonucleotide 391. For example, as illustrated in FIG. 3B, the oligonucleotide of label 131 may disrupt the non-covalent association between oligonucleotides 381, 391 by hybridizing to oligonucleotide 391. In a similar manner as described with reference to FIG. 2B, the kinetic association between oligonucleotides 131, 391 may be adjusted in such a manner to favor the displacement of oligonucleotide 381. For example, as illustrated in FIG. 3A, oligonucleotide 391 may include a toehold overhang T that provides a single-stranded region for oligonucleotide 131 to initiate the displacement of oligonucleotide 381. Other examples of ways in which to provide a higher Tm of the duplex between oligonucleotides 131 and 391 relative to the duplex of oligonucleotides 381 and 391 are provided above. As a result of disruption between oligonucleotides 381 and 391, fluorophore 161 may be sufficiently separated from quencher 171 as to fluoresce, as suggested in FIG. 3B by the lightened star. After polymerase 105 finishes adding nucleotide 121 to a growing polynucleotide (not specifically illustrated), then label 131 may be cleaved and may dissociate from oligonucleotide 391, leaving oligonucleotide 391 available to rehybridize to oligonucleotide 381. As a result of such rehybridizing, fluorophore 161 may be sufficiently close to quencher 171 as substantially not to fluoresce.

In other examples, each of the fluorophores and respective quencher is coupled to a respective hairpin oligonucleotide having first and second stem sequences and a loop sequence, and the non-covalent associations comprise hybridizations between the first and second stem sequences. For example, FIGS. 4A-4B schematically illustrate example compositions and operations in another process flow for sequencing using fluorophores and quenchers. In the simplified example shown in FIG. 4A (in which fluorophores 162, 163, 164 and quenchers 172, 173, 174 are omitted for clarity), fluorophore 161 and quencher 171 are coupled to hairpin oligonucleotide 481 which is coupled to the substrate via a linker which may be coupled to stem sequence 482 or stem sequence 483. Fluorophore 161 may be coupled to stem sequence 482 and quencher 171 may be coupled to stem sequence 483 that is hybridized to stem sequence 482, thus inhibiting fluorescence of fluorophore 161 as suggested by the darkened star. Alternatively, fluorophore 161 may be coupled to stem sequence 483 and quencher 171 may be coupled to stem sequence 482. Loop sequence 484 may be between stem sequences 482, 483. Each of the labels may include a respective oligonucleotide. In the simplified example shown in FIG. 4A (in which nucleotides 122, 123, 124 and labels 132, 133, 134 are omitted for clarity), label 131 of nucleotide 121 includes an oligonucleotide having a sequence that is complementary to loop sequence 484. For example, as illustrated in FIG. 4B, the oligonucleotide of label 131 may disrupt the non-covalent association between stem sequences 482, 483 by hybridizing to loop sequence 484. In some examples, the hybridization between oligonucleotide 131 and loop sequence 484 may form dsDNA which is substantially linear and thus facilitates separation between fluorophore 161 and quencher 171. As a result of the disruption between stem sequences 483, 483, fluorophore 161 may be sufficiently separated from quencher 171 as to fluoresce, as suggested in FIG. 4B by the lightened star. After polymerase 105 finishes adding nucleotide 121 to a growing polynucleotide (not specifically illustrated), then label 131 may be cleaved and may dissociate from loop sequence 484, such that stem sequences 482, 483 may rehybridize to one another. As a result of such rehybridizing, fluorophore 161 may be sufficiently close to quencher 171 as substantially not to fluoresce.

The sequence and length of loop sequence 484, and the sequences and lengths of stem sequences 482, 483 may affect the kinetics and thermodynamics of hybridization and disassociation between (a) stem sequences 482 and 483 with one another, and (b) label 131 and loop sequence 484. In some examples, stem sequences 482 and 483 may be designed so as to have a ΔG₀ of about −1.5 to about −2.0 kcal/mol. In some examples, the Tm of hybridization between stem sequences 482 and 483 may be increased by decreasing the length of loop sequence 484. In some examples, stem sequences 482 and 483 may have lengths of about 6 or fewer bases, e.g., 4, 5, or 6 bases. In some examples, stem sequences 482 and 483 may have a relatively high GC content. For example, hairpin oligonucleotide 481 may have a composition such as CCGCGC-loop-GCGCGG where CCGCGC corresponds to stem sequence 482 and GCGCGG corresponds to stem sequence 483.

It will be appreciated that oligonucleotides such as described with reference to FIGS. 2A-2B, 3A-3B, and 4A-4B may include, but are not limited to DNA, PNA, LNA, RNA, and combinations thereof. For example, one or more oligonucleotides in a composition may include DNA, and one or more other oligonucleotides in the composition may include PNA or LNA. Other example modifications that may impact relative Tms of different duplexes are provided further above. Additionally, it will be appreciated that schemes such as described with reference to FIGS. 2A-2B, 3A-3B, and 4A-4B may be adapted for use with polymers other than oligonucleotides. For example, the oligonucleotides may be replaced by peptides, and interactions between the oligonucleotides may be replaced by protein-protein interactions or peptide-peptide interactions, such as coiled coils, leucine zippers, barnase-barstar, Ras-Raf, or other ligand-receptor interactions.

As noted further above, some examples may include quenchers, fluorophores, or both quenchers and fluorophores, that are in solution. FIGS. 5A-5B schematically illustrate example compositions and operations in another process flow for sequencing using fluorophores and quenchers. In the non-limiting example shown in FIG. 5A, composition 500 includes substrate 501 and polymerase 505 coupled to the substrate similarly as described with reference to FIG. 1A. Composition 500 further may include solution 500 that includes (i) quenchers 570 and (ii) nucleotides 521, 522, 523, 524 coupled to respective labels 531, 532, 533, 534. In the illustrated example, each of label 531, 532, 533, 534 may be coupled to a respective fluorophore 561, 562, 563, 564. Each fluorophore 561, 562, 563, 564 may be non-covalently associated with at least one of the quenchers 570, thus inhibiting fluorescence of the fluorophores as suggested by the darkened stars. Composition 500 further may include an element coupled to the substrate to disrupt the non-covalent associations between the fluorophores and the quenchers. For example, element 581 coupled to substrate may disrupt non-covalent associations between fluorophores 561, 562, 563, 564 and quenchers 570, thus sufficiently separating the fluorophores from the quenchers that the fluorophores may fluoresce.

Illustratively, in a manner similar to that described with reference to FIGS. 1A-1B, polymerase 505 may be to add the nucleotides 521, 522, 523, 524 to first polynucleotide 540 using at least a sequence of second polynucleotide 550. At the particular time illustrated in FIG. 5B, polymerase 505 is acting upon nucleotide 521 (illustratively, G) which maintains label 531 at a location that is sufficiently close to element 581 that element 581 may separate fluorophore 561 from quencher 571. Accordingly, as suggested in FIG. 5B by the lightened star, fluorophore 561 may fluoresce in response to suitable optical excitation, and optical detection circuitry 580 may detect fluorescence from the fluorophore resulting from the disruption in the non-covalent association.

At another time (not specifically illustrated), polymerase 505 may act upon nucleotide 522 (illustratively, T) which maintains label 532 at a location that is sufficiently close to element 581 that element 581 may separate fluorophore 562 from quencher 572, such that fluorophore 562 may fluoresce in response to suitable optical excitation. At another time (not specifically illustrated), polymerase 505 may act upon nucleotide 523 (illustratively, A) which maintains label 533 at a location that is sufficiently close to element 581 that element 581 may separate fluorophore 562 from quencher 572, such that fluorophore 562 may fluoresce in response to suitable optical excitation. At another time (not specifically illustrated), polymerase 505 may act upon nucleotide 524 (illustratively, C) which maintains label 534 at a location that is sufficiently close to element 581 that element 581 may separate fluorophore 562 from quencher 572, such that fluorophore 562 may fluoresce in response to suitable optical excitation. Fluorophores 561, 562, 563, 564 may emit fluorescence of different wavelengths than one another, e.g., may be different types of fluorophores than each other, so that nucleotides 521, 522, 523, 524 may be distinguished from one another using the respective fluorescence from those fluorophores (e.g., as detected using optical detection circuitry 580). For example, nucleotide 521 may be identified using fluorescence from fluorophore 561, nucleotide 522 may be identified using fluorescence from fluorophore 562, nucleotide 523 may be identified using fluorescence from fluorophore 563, and nucleotide 524 may be identified using fluorescence from fluorophore 564. Quencher 570 suitably may be selected so as to selectively and non-covalently associate with, fluorophores 561, 562, 563, 564, and to respectively inhibit fluorescence therefrom.

In some examples, each of labels 531, 532, 533, 534 may include or may be coupled to a respective first oligonucleotide to which a respective fluorophore 561, 562, 563, 564 is coupled, each of the quenchers 570 is coupled to a respective second oligonucleotide, and the non-covalent associations comprise hybridizations between the first oligonucleotides and the second oligonucleotides. For example, fluorophores 561, 562, 563, 564 may be coupled to first oligonucleotides (or other polymers, such as peptides) in a manner similar to that described with reference to FIGS. 2A-2B and 3A-3B, and such oligonucleotides respectively may be coupled to nucleotides 521, 522, 523, 524. Quenchers 570 may be coupled to second oligonucleotides (or other polymers, such as peptides) in a manner similar to that described with reference to FIGS. 2A-2B and 3A-3B, and such oligonucleotides may be in solution 520. The first oligonucleotides to which fluorophores 561, 562, 563, 564 are coupled may, in some examples, have the same sequence as one another, and such sequence may be complementary to that of the second oligonucleotides to which quenchers 570 are coupled such that the first and second oligonucleotides hybridize to each other in solution. Element 581 coupled to the surface may include a third oligonucleotide (or other polymer, such as a peptide). Oligonucleotide 581 may disrupt the non-covalent associations by hybridizing to the first oligonucleotide, e.g., may have a sequence that is complementary to the first oligonucleotide and displaces the first oligonucleotide's hybridization to the second oligonucleotide, to which quencher 570 is coupled. Alternatively, oligonucleotide 581 may disrupt the non-covalent associations by hybridizing to the second oligonucleotide, e.g., may have a sequence that is complementary to the second oligonucleotide and displaces the first oligonucleotide's hybridization to the second oligonucleotide, to which quencher 570 is coupled. As a result of such disruptions, fluorophores 561, 562, 563, 564 respectively may fluoresce, and optical detection circuitry 580 may detect such fluorescence for use in identifying respective nucleotides 521, 522, 523, 524 as polymerase 505 adds them to growing polynucleotide 540. After polymerase adds respective nucleotides 521, 522, 523, 524 to the growing polynucleotide 540, new non-covalent associations may form between fluorophores 561, 562, 563, 564 and respective quenchers 570 in the solution, thus inhibiting fluorescence unless and until polymerase 505 acts upon another nucleotide.

Although quenchers 570 may be in solution in some examples, the quenchers need not necessarily be free-floating and independent of fluorophores 561, 562, 563, 564 in a manner such as suggested in FIGS. 5A-5B. In other examples, each of the fluorophores and respective quencher is coupled to a respective hairpin oligonucleotide having first and second stem sequences and a loop sequence, and the non-covalent associations comprise hybridizations between the first and second stem sequences. For example, in a manner such as described with reference to FIGS. 4A-4B, fluorophores 561, 562, 563, 564 may be coupled to a first stem sequence of a hairpin oligonucleotide, a quencher 570 may be coupled to a second stem sequence of the hairpin oligonucleotide that is complementary to, and hybridizes to, the first stem sequence. The first and second stem sequences may be separated from one another by a loop sequence. The hairpin oligonucleotides respectively may be coupled to nucleotides 521, 522, 523, 524. Element 581 coupled to the surface may include an oligonucleotide. Oligonucleotide 581 may be to disrupt the non-covalent associations by hybridizing to the loop sequence, e.g., may have a sequence that is complementary to the loop sequence and separates the first stem sequence, to which one of fluorophores 561, 562, 563, 564 is coupled, from the second stem sequence, to which quencher 570 is coupled, in a manner similar to that described with reference to FIGS. 4A-4B. As a result of such disruptions, fluorophores 561, 562, 563, 564 respectively may fluoresce, and optical detection circuitry 580 may detect such fluorescence for use in identifying respective nucleotides 521, 522, 523, 524 as polymerase 505 adds them to growing polynucleotide 540. After polymerase adds respective nucleotides 521, 522, 523, 524 to the growing polynucleotide 540, new non-covalent associations may form between fluorophores 561, 562, 563, 564 and respective quenchers 570 in the solution (e.g., coupled to the hairpin oligonucleotide), thus inhibiting fluorescence unless and until polymerase 505 acts upon another nucleotide.

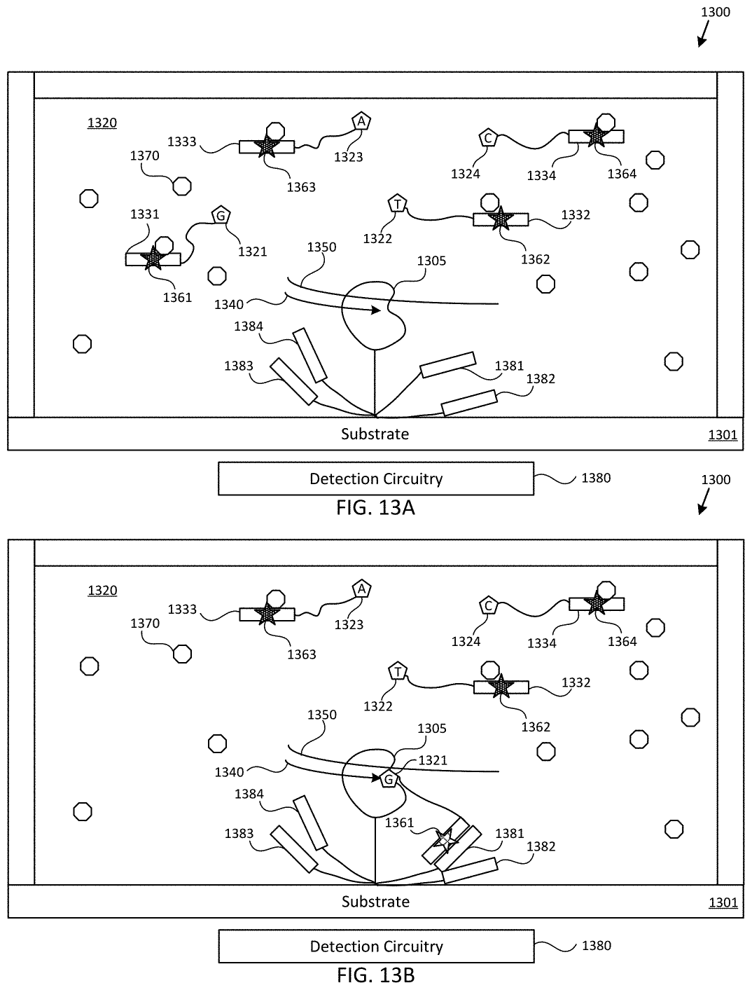

Although FIGS. 5A-5B may suggest a composition in which a single element 581 is coupled to substrate 501, it will be appreciated that any suitable number of elements may be coupled to the substrate, e.g., a plurality of elements 581. For example, FIGS. 13A-13B schematically illustrate example compositions and operations in a process flow for sequencing using fluorophores and quenchers. In the example shown in FIG. 13A, each of the fluorophores 1361, 1362, 1363, 1364 may be coupled to, a respective first oligonucleotide 1331, 1332, 1333, 1334 coupled to a respective nucleotide 1321, 1322, 1323, 1324, e.g., in a manner similar to that described above. Fluorophores 1361, 1362, 1363, 1364 may be or include different types of fluorophores than one another. First oligonucleotides 1331, 1332, 1333, 1334 may have the same sequences as one another, or may have different sequences than one another. In a manner similar to that described with reference to FIGS. 5A-5B, quenchers 1370 may be coupled to second oligonucleotides (or other polymers, such as peptides) in a manner similar to that described with reference to FIGS. 2A-2B and 3A-3B, and such oligonucleotides may be in solution 1320. The first oligonucleotides 1331, 1332, 1333, 1334 to which fluorophores 1361, 1362, 1363, 1364 respectively are coupled may, in some examples, have the same sequence as one another, and such sequence may be complementary to that of the second oligonucleotides to which quenchers 1370 are coupled such that the first and second oligonucleotides hybridize to each other in solution 1320. Composition 1300 may include a plurality of elements each including a respective third oligonucleotide 1381, 1382, 1383, 1384 coupled to the substrate 1301. Each of the first oligonucleotides 1331, 1332, 1333, 1334 may hybridize to a corresponding one of the third oligonucleotides 1381, 1382, 1383, 1384 while polymerase 1305 adds the corresponding nucleotide 1321, 1322, 1323, 1324 to first polynucleotide 1340 using the sequence of second polynucleotide 1350, and the third oligonucleotide may cause quencher 570 to disassociate from the respective fluorophore. For example, at the particular time illustrated in FIG. 13B, third oligonucleotide 1381 hybridizes to first oligonucleotide 1331, and thus displaces quencher 1370, allowing fluorophore 1361 to fluoresce and thus be detected by detection circuitry 1380.

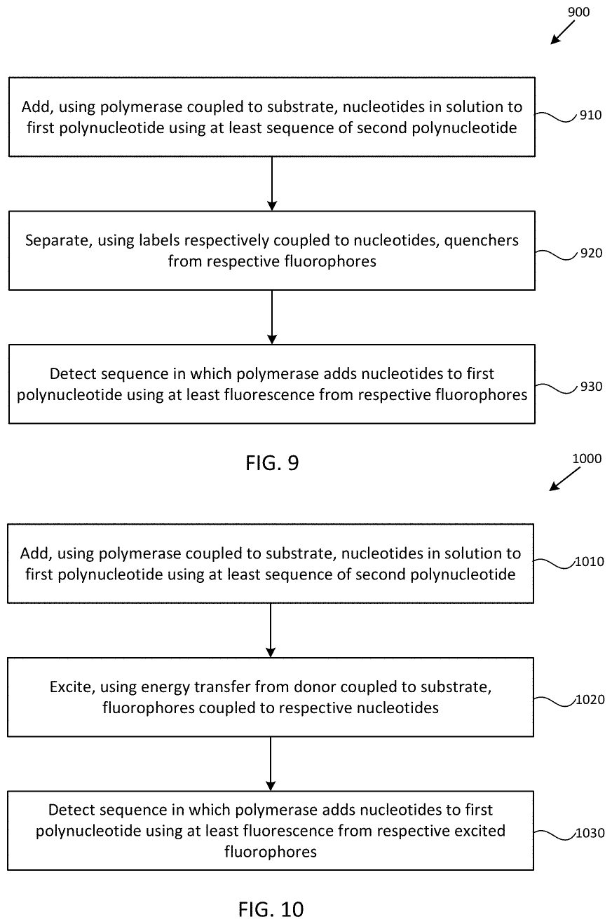

FIG. 9 illustrates an example flow of operations in a method 900 for sequencing using fluorophores and quenchers. Method 900 may include adding, using a polymerase coupled to a substrate, nucleotides in a solution to a first polynucleotide using at least a sequence of a second polynucleotide (operation 910). For example, polymerase 105 may add nucleotides 121, 122, 123, 124 in solution 120 to polynucleotide 140 using at least the sequence of polynucleotide 150, in a manner such as described with reference to FIGS. 1A-1B, or polymerase 505 may add nucleotides 521, 522, 523, 524 in solution 520 to polynucleotide 540 using at least the sequence of polynucleotide 550, in a manner such as described with reference to FIGS. 5A-5B. Method 900 further may include separating, using labels respectively coupled to the nucleotides, quenchers from respective fluorophores (operation 920). For example, labels 131, 132, 133, 134 may separate quenchers 171, 172, 173, 174 from respective fluorophores 161, 162, 163, 164 in a manner such as described with reference to FIGS. 1A-1B, or labels 531, 532, 533, 534 may separate quenchers 570 from respective fluorophores 561, 562, 563, 564 in a manner such as described with reference to FIGS. 5A-5B, or labels 1331, 1332, 1333, 1334 may separate quenchers 1370 from respective fluorophores 1361, 1362, 1363, 1364 in a manner such as described with reference to FIGS. 13A-13B. Method 900 further may include detecting a sequence in which the polymerase adds the nucleotides to the first polynucleotide using at least fluorescence from the respective fluorophores (operation 930). For example, optical detection circuitry 180 may detect fluorescence from fluorophores 161, 162, 163, 164 as polymerase 105 adds respective nucleotides 121, 122, 123, 124 to polynucleotide 140 in a manner such as described with reference to FIGS. 1A-1B, or optical detection circuitry 580 may detect fluorescence from fluorophores 561, 562, 563, 564 as polymerase 505 adds respective nucleotides 521, 522, 523, 524 to polynucleotide 540 in a manner such as described with reference to FIGS. 5A-5B, or optical detection circuitry 1380 may detect fluorescence from fluorophores 1361, 1362, 1363, 1364 as polymerase adds respective nucleotides 1321, 1322, 1323, 1324 to polynucleotide 1340 in a manner such as described with reference to FIGS. 5A-5B.

Example Methods for Sequencing Using Fluorophores and Donors, and Associated Compositions

In some examples provided herein, polynucleotides may be sequenced on a single-molecule basis using fluorophores and donors. For example, a polymerase coupled to a substrate may add nucleotides in a solution to a first polynucleotide using at least a sequence of a second (“target”) polynucleotide. Fluorophores coupled to respective nucleotides may be excited, using energy transfer from a donor coupled to the substrate. In this regard, the fluorophores also may be referred to as acceptors. A sequence in which the polymerase adds the nucleotides to the first polynucleotide may be determined using at least fluorescence from the respective excited fluorophores.

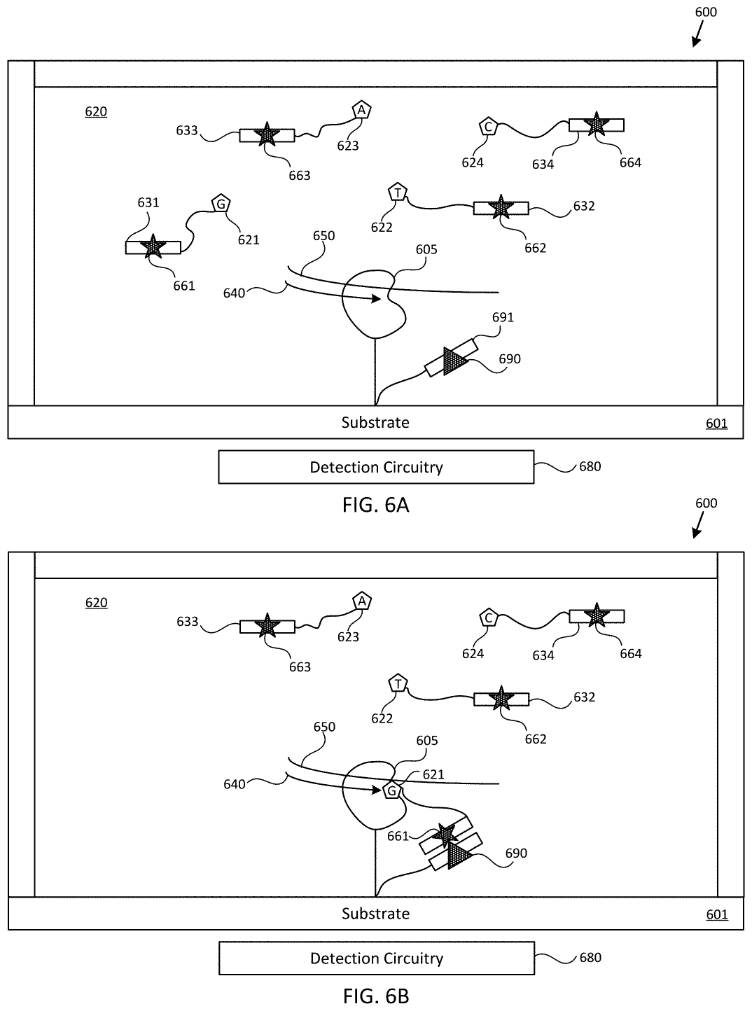

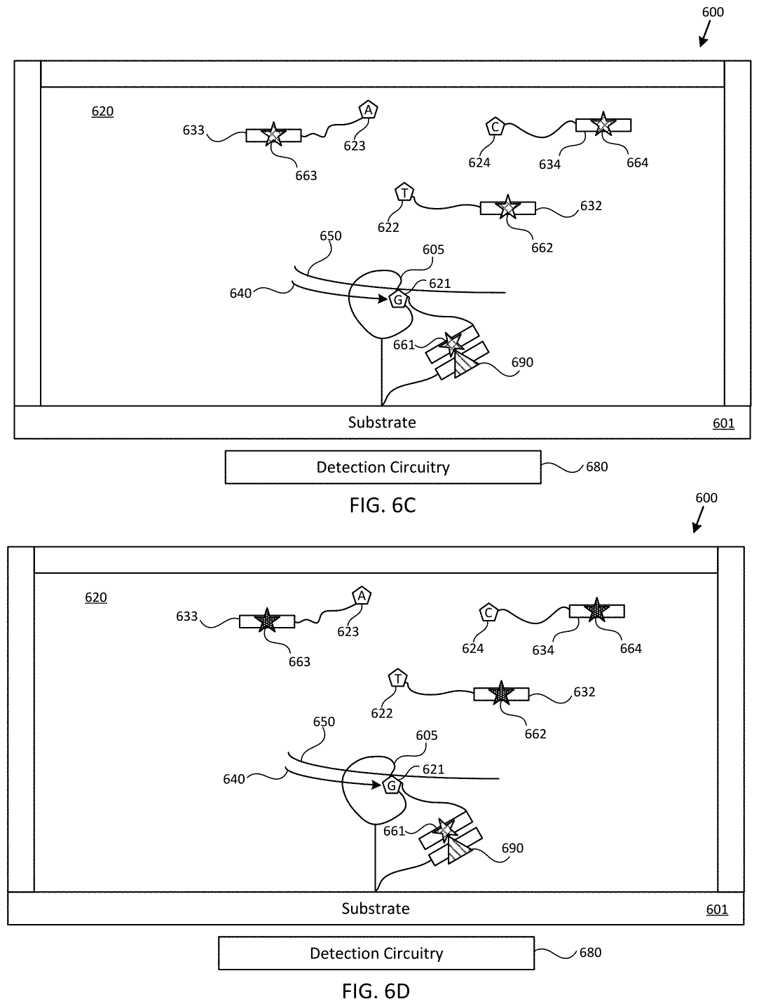

For example, FIGS. 6A-6D schematically illustrate example compositions and operations in a process flow for sequencing using fluorophores and donors. Referring now to FIG. 6A, composition 600 may include substrate 601, polymerase 605 coupled to the substrate, donor 690 coupled to the substrate, and solution 620 including nucleotides 621, 622, 623, 624 coupled to respective fluorophores 661, 662, 663, 664. For example, nucleotides 621, 622, 623, 624 may be coupled to respective labels 631, 632, 633, 634 to which fluorophores 661, 662, 663, 664 respectively may be coupled. Donor 690 may be coupled to an element 691 that is coupled via a linker to substrate 601. Composition 600 further may include optical detection circuitry 680. In a manner similar to that described elsewhere herein, polymerase 605 may be to add nucleotides 621, 622, 623, 624 to first polynucleotide 640 using at least a sequence of second polynucleotide 650. Optical detection circuitry 680 may be to detect fluorescence from the fluorophores 661, 662, 663, 664 resulting from energy transfer from donor 690 to the fluorophores coupled to the respective nucleotides. For example, at the particular time illustrated in FIG. 6B, polymerase 605 is acting upon nucleotide 621 (illustratively, G) which maintains label 631 at a location that is sufficiently close to donor 690 that donor 690 may transfer energy to fluorophore 661. Accordingly, fluorophore 661 may fluoresce in response to suitable optical excitation of donor 690, and optical detection circuitry 680 may detect fluorescence from the fluorophore resulting from transfer of energy from donor 690 to fluorophore 661.

For example, composition 600 further may include an excitation light source to optically excite donor 690 at a first time, and the optically excited donor may excite fluorophore 661 using the energy transfer. At the particular time illustrated in FIG. 6B, donor 690 has not yet been optically excited, as suggested by the darkened triangle, and so energy has not yet transferred from donor 690 to fluorophore 661 so as to cause fluorophore 661 to fluoresce, as suggested by the darkened star. At the particular time illustrated in FIG. 6C, donor 690 is optically excited, as suggested by the lightened triangle, and energy transfers from donor 690 to fluorophore 661, as a result of which fluorophore 661 may fluoresce, as suggested by the lightened star. In some examples, optically exciting the donor 690 at the first time also may optically excite fluorophores 662, 663, 664 coupled to the respective nucleotides 622, 623, 624, as illustrated in FIG. 6C, as suggested by the lightened star. Fluorophores 661, 662, 663, 664 may have a first emission lifetime after the optical excitation, and donor 690 may have a second emission lifetime after the optical excitation that is longer than the first emission lifetime. This difference in emission lifetimes may be used to select periods in which optical detection circuitry 680 may selectively detect fluorescence from fluorophore 661 coupled to nucleotide 621 upon which polymerase 605 is acting, while inhibiting detection of fluorescence from fluorophores 662, 663, 664 which are in solution 620.

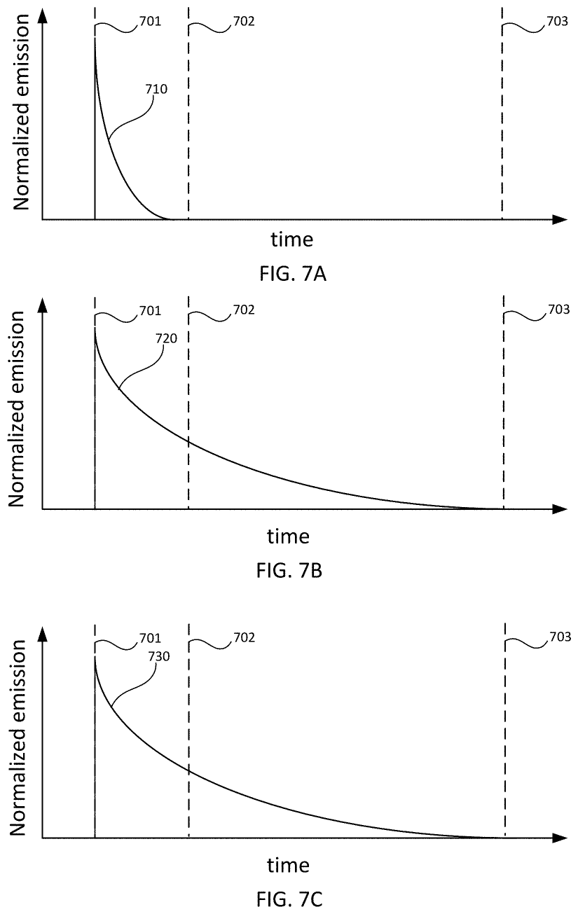

For example, FIGS. 7A-7C schematically illustrate example emission lifetimes in a process flow for sequencing using fluorophores and donors. FIG. 7A illustrates example emission lifetimes 710 of fluorophores within composition 600 resulting from an appropriately pulsed optical excitation with a duration, for example of less than about 10 nsec, less than about 50 nsec, or less than about 100 nsec, at first time 801, e.g., of fluorophores 661, 662, 663, 664 by an excitation light source, regardless of whether such fluorophores are coupled to nucleotides in solution 620 or are coupled to a nucleotide being acted upon by polymerase 605. It may be understood from FIG. 7A that fluorescence (emission) from fluorophores 661, 662, 663, 664 resulting from optical excitation is substantially complete by second time 702. In comparison, FIG. 7B illustrates an example emission lifetime 720 of donor 690 resulting from optical excitation at first time 701. It may be understood from FIG. 7B that energy emission from donor 690 resulting from optical excitation is chosen to be significantly longer than the energy emission from fluorophores 661, 662, 663, 664 resulting from the same optical excitation, and is substantially complete by third time 703. At least a portion of such energy emission from donor 690 may transfer to fluorophore 661 when polymerase 605 is acting upon nucleotide 621 in a manner such as illustrated in FIGS. 6A-6C, and as such fluorophore 661 may continue to fluoresce even after fluorophores in solution 620 stop fluorescing. The ability to resolve the fluorescence of 661 permits an improvement in signal to noise and overcomes the bulk fluorescence of fluorophores in solution 620. A time-resolved measurement allows for suppression of short-lived emission due to background fluorescence and directly excited acceptors.

For example, FIG. 7C illustrates an example emission lifetime 730 of fluorophore 661 resulting from energy transfer from donor 690, e.g., while polymerase 605 is acting upon nucleotide 621. It may be understood from FIG. 7C that energy emission from fluorophore 661 resulting from energy transfer from donor 690 is chosen to be significantly longer than the energy emission from fluorophores 661, 662, 663, 664 resulting from the same optical excitation, and is substantially complete by third time 703. Accordingly, so as to selectively detect fluorescence from fluorophore 661 coupled to nucleotide 621 upon which polymerase 605 is acting, while inhibiting detection of fluorescence from fluorophores 662, 663, 664 which are in solution 620, fluorophore 661 (and thus the next nucleotide in the sequence being added to growing polynucleotide 640) is detected using fluorescence beginning at a second time 702 after the first emission lifetime 710 ends. Such detection may end, for example, at third time 703, or any other suitable time following collection of a sufficient amount of fluorescence to identify nucleotide 621. For example, as illustrated in FIG. 6D, fluorophore 661 continues to fluoresce, as suggested by the lightened star, as a result of energy transfer from excited donor 690, while fluorophores 662, 663, 664 substantially have stopped fluorescing, as suggested by the darkened stars, because a sufficient amount of time since their optical excitation has passed.

At another time (not specifically illustrated in FIGS. 6A-6D), polymerase 605 may act upon nucleotide 622 (illustratively, T) which maintains label 632 at a location that is sufficiently close to donor 690 such that fluorophore 662 may fluoresce in response to energy transfer from donor 690 following suitable optical excitation. At another time (not specifically illustrated), polymerase 605 may act upon nucleotide 623 (illustratively, A) which maintains label 633 at a location that is sufficiently close to donor 690 such that fluorophore 663 may fluoresce in response to energy transfer from donor 690 following suitable optical excitation. At another time (not specifically illustrated), polymerase 605 may act upon nucleotide 624 (illustratively, C) which maintains label 634 at a location that is sufficiently close to donor 690 such that fluorophore 664 may fluoresce in response to energy transfer from donor 690 following suitable optical excitation. Fluorophores 661, 662, 663, 664 may emit fluorescence of different wavelengths than one another, e.g., may be different types of fluorophores than each other, so that nucleotides 621, 622, 623, 624 may be distinguished from one another using the respective fluorescence from those fluorophores (e.g., as detected using optical detection circuitry 680). For example, nucleotide 621 may be identified using fluorescence from fluorophore 661, nucleotide 622 may be identified using fluorescence from fluorophore 662, nucleotide 623 may be identified using fluorescence from fluorophore 663, and nucleotide 624 may be identified using fluorescence from fluorophore 664. Donor 690 suitably may be selected so as to selectively and non-covalently associate with, fluorophores 661, 662, 663, 664, and to respectively transfer energy thereto so as to cause the fluorophores to fluoresce.

In some examples, each of labels 631, 632, 633, 634 may include a respective first oligonucleotide to which a respective fluorophore 661, 662, 663, 664 is coupled, and donor 690 is coupled to a respective second oligonucleotide (e.g., structure 691) which is coupled to substrate 600. Each of the first oligonucleotides 631, 632, 633, 634 may hybridize to the second oligonucleotide 691 while the polymerase adds that nucleotide to the first polynucleotide 640. For example, fluorophores 661, 662, 663, 664 may be coupled to first oligonucleotides (or other polymers, such as peptides) in a manner similar to that described with reference to FIGS. 2A-2B and 3A-3B, and such oligonucleotides respectively may be coupled to nucleotides 621, 622, 623, 624. Donor 690 may be coupled to a second oligonucleotide (or other polymer, such as a peptide) in a manner similar to that described with reference to FIGS. 2A-2B and 3A-3B, and such oligonucleotide may be coupled to substrate 601. The first oligonucleotides to which fluorophores 661, 662, 663, 664 are coupled may, in some examples, have the same sequence as one another, and such sequence may be complementary to that of the second oligonucleotides to which donor 690 is such that the first and second oligonucleotides hybridize to each other when polymerase 605 is acting upon a respective one of nucleotides 621, 622, 623, 624. As a result of such hybridization, energy transfers from the donor to that fluorophore 661, 662, 663, or 664 while the first oligonucleotide is hybridized to the second oligonucleotide, causing that fluorophore to fluoresce. Optical detection circuitry 680 may detect such fluorescence for use in identifying respective nucleotides 621, 622, 623, 624 as polymerase 605 adds them to growing polynucleotide 640. After polymerase adds respective nucleotides 621, 622, 623, 624 to the growing polynucleotide 640, fluorophores 661, 662, 663, 664 may disassociate from donor 690, thus inhibiting fluorescence unless and until polymerase 605 acts upon another nucleotide.

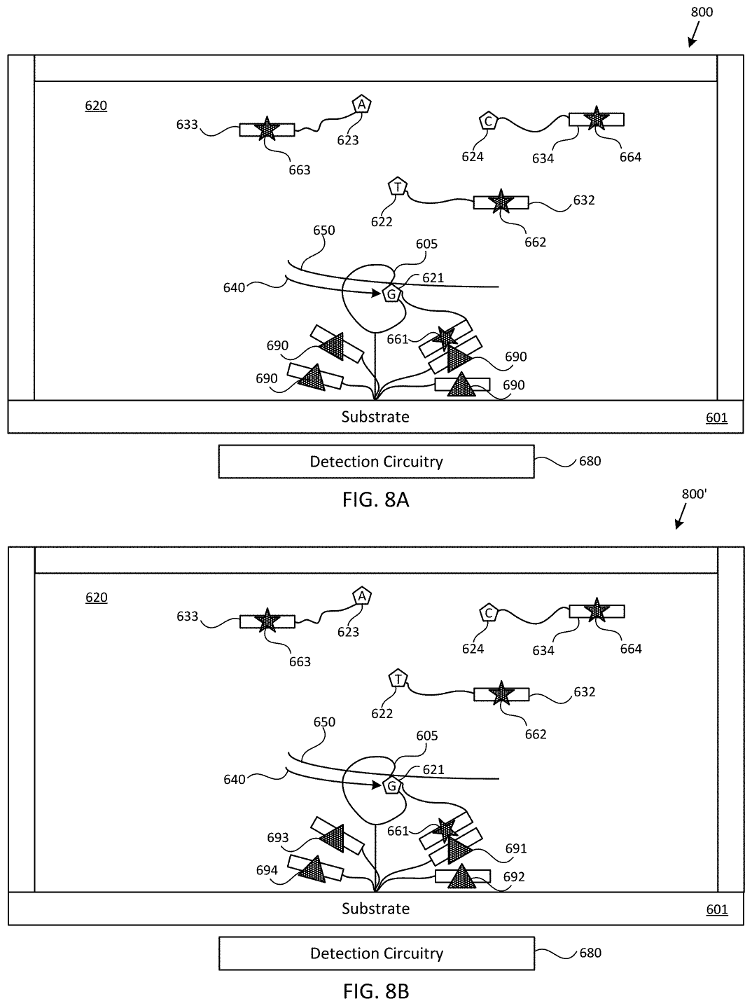

Although FIGS. 6A-6D may suggest a composition in which a single donor is coupled to substrate 601, it will be appreciated that any suitable number of donors may be coupled to the substrate, e.g., a plurality of donors, which may be the same as one another or may be different than one another. FIGS. 8A-8B schematically illustrates example compositions and operations in a process flow for sequencing using fluorophores and quenchers. In the example illustrated in FIG. 8A, each of the fluorophores 661, 662, 663, 664 may be coupled to a respective first oligonucleotide coupled to a respective nucleotide 621, 622, 623, 624, e.g., in a manner similar to that described above. Composition 800 may include a plurality of donors 690 each coupled to a respective second oligonucleotide coupled to the substrate 601. Each of the first oligonucleotides may have the same sequence, and each of the second oligonucleotides may have the same sequence which is complementary to that of the first oligonucleotides. As such, the first oligonucleotide may hybridize to any given one of the second oligonucleotides while the polymerase adds the respective nucleotide to the first polynucleotide, and energy may transfer from the respective donor 690 to that fluorophore 661, 662, 663, 664 while the first oligonucleotide is hybridized to the second oligonucleotide in a manner such as illustrated in FIG. 8A. The fluorophores 661, 662, 663, 664 may be the same as one another (e.g., may fluoresce at the same wavelength as one another, in a one-dye scheme), or may be different than one another (e.g., may fluoresce at different wavelengths than one another, in a multiple-dye scheme such as a two-dye or four-dye scheme). The fluorophores 661, 662, 663, 664 each may fluoresce responsive to energy transferred from donor 690. For example, each of the donors 690 may include terbium or another rare earth element and the fluorophores 661, 662, 663, 664 independently may be selected from the group consisting of fluorescein, Alexa Fluor 488, GFP, YFP, rhodamine, Alexa Fluor 546, Alexa Fluor 633, and Alexa Fluor 647.

Alternatively, in the example illustrated in FIG. 8B, fluorophore 661 may be coupled to a first oligonucleotide coupled to nucleotide 621, fluorophore 662 may be coupled to a second oligonucleotide coupled to nucleotide 622, fluorophore 663 may be coupled to a third oligonucleotide coupled to nucleotide 623, and fluorophore 664 may be coupled to a fourth oligonucleotide coupled to nucleotide 624. Fluorophores 661, 662, 663, 664 may be different than one another, e.g., may fluoresce at different wavelengths than one another. The first, second, third, and fourth oligonucleotides may have different sequences than one another. Composition 800′ may include donor 691 coupled to a fifth oligonucleotide coupled to the substrate 601, donor 692 coupled to a sixth oligonucleotide coupled to the substrate 601, donor 693 coupled to a seventh oligonucleotide coupled to the substrate 601, and donor 694 coupled to an eighth oligonucleotide coupled to the substrate. Donors 691, 692, 693, 694 may be different than one another, e.g., may transfer energy at different wavelengths than one another. The fifth, sixth, seventh, and eight oligonucleotides may have different sequences than one another. The sequence of the fifth oligonucleotide may be complementary to that of the first oligonucleotide, the sequence of the sixth oligonucleotide may be complementary to that of the second oligonucleotide, the sequence of the seventh oligonucleotide may be complementary to that of the third oligonucleotide, and sequence of the eight oligonucleotide may be complementary to that of the fourth oligonucleotide.

As such, the first oligonucleotide may hybridize to the fifth oligonucleotide while the polymerase 605 adds the respective nucleotide 621 to the first polynucleotide, and energy may transfer from the respective donor 691 to that fluorophore 661 while the first oligonucleotide is hybridized to the fifth oligonucleotide in a manner such as illustrated in FIG. 8A, responsive to which fluorophore 661 fluoresces. Similarly, the second oligonucleotide may hybridize to the sixth oligonucleotide while the polymerase 605 adds the respective nucleotide 622 to the first polynucleotide, and energy may transfer from the respective donor 692 to that fluorophore 662 while the second oligonucleotide is hybridized to the sixth oligonucleotide in a manner similar to that illustrated in FIG. 8A, responsive to which fluorophore 662 fluoresces. Similarly, the third oligonucleotide may hybridize to the seventh oligonucleotide while the polymerase 605 adds the respective nucleotide 623 to the first polynucleotide, and energy may transfer from the respective donor 693 to that fluorophore 663 while the third oligonucleotide is hybridized to the seventh oligonucleotide in a manner similar to that illustrated in FIG. 8A, responsive to which fluorophore 663 fluoresces. Similarly, the fourth oligonucleotide may hybridize to the eighth oligonucleotide while the polymerase 605 adds the respective nucleotide 624 to the first polynucleotide, and energy may transfer from the respective donor 694 to that fluorophore 664 while the fourth oligonucleotide is hybridized to the eight oligonucleotide in a manner similar to that illustrated in FIG. 8A, responsive to which fluorophore 664 fluoresces. The nucleotides 621, 622, 623, 624 may be identified using the resulting fluorescence from the respective fluorophore 661, 662, 663, 664.

Any suitable combination(s) of donors and fluorophores may be used in examples such as described with reference to FIGS. 6A-6D, 7A-7C, and 8A-8B. For example, donor 690 may include a rare earth element, which also may be referred to as a lanthanide. Example rare earth elements that may be included in donor 690 include La, Ce, Pr, Nd, Pm, Sm, Eu, Gd, Tb, Dy, Ho, Er, Tm, Yb, and Lu. Specific examples of lanthanide donors that may be included in donor 690 include Sm(III), Eu(III), Tb(III), and Dy(III). The donor 690 (e.g., rare earth element) may be provided in any suitable form. For example, the donor 690 may include a nanoparticle (illustratively, having a diameter of about 1 nm to about 30 nm, or about 5 nm to about 20 nm). Or, for example, the donor may include a rare earth element chelate or cryptate. Non-limiting examples of donor-acceptor pairs, which may be referred to as FRET pairs, include: terbium (donor) and TAMRA, BODIPY, GFP, Alexa Fluor 488, YFP, rhodamine, Alexa Fluor 546, Alexa Fluor 633, or Alexa Fluor 647, FAM, 5FAM, 5TAMRA, TMR or fluorescein (acceptors); europium chelate (donor) and Alexa Fluor 647 or Cy5 (acceptors); Cy3 (donor) and Cy5 (acceptor); Eu (III)-chelate nanoparticle (donor) and quantum dot, Alexa Fluor680 (acceptor); cryptate (donor) and XL665 (acceptor); and terbium or europium (donor) and a quantum dot, such as a CdSe/ZnS core/shell quantum dot (acceptor).

FIG. 10 illustrates an example flow of operations in a method 1000 for sequencing using fluorophores and donors. Method 1000 may include adding, using a polymerase coupled to a substrate, nucleotides in a solution to a first polynucleotide using at least a sequence of a second polynucleotide (operation 1010). For example, polymerase 605 may add nucleotides 621, 622, 623, 624 in solution 620 to polynucleotide 640 using at least the sequence of polynucleotide 6150, in a manner such as described with reference to FIGS. 6A-6D. Method 600 further may include exciting, using energy transfer from a donor coupled to the substrate, fluorophores coupled to respective nucleotides (operation 1020). For example, labels 631, 632, 633, 634 coupled to respective fluorophores 661, 662, 663, 664 may come in sufficiently close proximity to donor 690 that donor 690 may transfer energy thereto, in a manner such as described with reference to FIGS. 6A-6D. Method 1000 further may include detecting a sequence in which the polymerase adds the nucleotides to the first polynucleotide using at least fluorescence from the respective excited fluorophores (operation 1030). For example, optical detection circuitry 680 may detect fluorescence from fluorophores 661, 662, 663, 664 as polymerase 605 adds respective nucleotides 621, 622, 623, 624 to polynucleotide 640 in a manner such as described with reference to FIGS. 6A-6D.

Example Nucleotides, Polymerases, and Reaction Conditions

It will be appreciated that in examples such as provided herein, the nucleotides, polymerases, and reaction conditions suitably may be selected so as to accurately sequence the target polynucleotide. In some examples, detection of a fluorescent signal may occur once a polymerase acts upon a labeled nucleotide (interchangeably referred to herein as, for example, a complexation condition, a non-incorporating condition, and a pause of catalysis), but prior to actual nucleotide incorporation. This aspect utilizes controlled catalysis in which the chemical incorporation of a nucleotide is slowed, paused, or temporarily inhibited so as to detect the signal and call the correct base during a complexation condition, before the incorporation is allowed to proceed (interchangeably referred to herein as, for example, a polymerization condition, an incorporating condition, and/or a catalytic condition) so that another nucleotide may be incorporated.

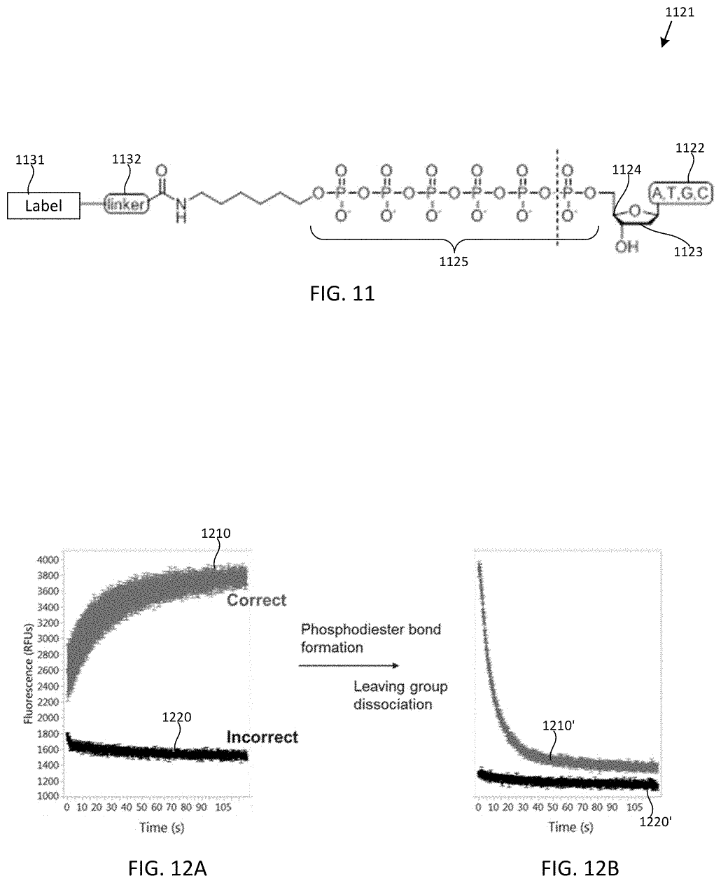

In some examples, the nucleotides include a compound of Formula (I):

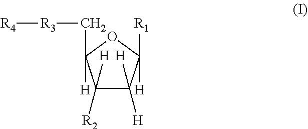

wherein R₁ includes a nitrogenous base selected from adenine, guanine, cytosine, thymine and uracil; R₂ includes —OH, —OZ, —H, or —Z, where Z is a removable protecting group comprising an azido group; R₃ includes a linker including three or more phosphate groups (e.g., four or more, five or more, or six or more phosphate groups); and R₄ includes a label such as described with reference to any of FIG. 1A-1B, 2A-2B, 3A-3B, 4A-4B, 5A-5B, 6A-6D, 7A-7D, 8A-8B, 9, or 10. FIG. 11 illustrates an example labeled nucleotide that may be used in the present compositions and methods. Nucleotide 1121 illustrated in FIG. 11 may include nitrogenous base 1122, sugar 1123, hexaphosphate group 1125 coupled to the 5′-position 1124 of sugar 1123, and linker 1132 coupling label 1131 to hexaphosphate group 1125, e.g., may be one nonlimiting example of a nucleotide of Formula (I).

Changes in conditions may facilitate the transition from complexation conditions to polymerization conditions. Alternatively, the complexation and polymerization may be performed in a single pot, under a single condition (e.g., at a single temperature and in a solution including all components required for both complexation and polymerization), so as to avoid the need for changes in conditions. In the presence of a catalytic condition, a DNA polymerase may incorporate the nucleotide to the DNA, causing dissociation of a leaving group (e.g., 5′-polyphosphate of the nucleotide), which may carry with a label. In some examples, nucleotides may include a 3′ reversible terminator (e.g., azido group) in addition to a 5′ terminal phosphate label.

The complexation condition as described herein refers to a condition effective to form a complex. Such condition also may be, but need not necessarily be, effective to form polymerization. Detection of a fluorescent signal may occur once a free nucleotide and a polymerase are bound to complementary polynucleotide (e.g., polynucleotide 140, 540, or 640), opposite to the template polynucleotide (e.g., polynucleotide 150, 550, or 650), but prior to actual nucleotide incorporation (this complex that is formed prior to nucleotide incorporation is referred to herein as, for example, a complexation condition). A complexation condition as described herein may utilize controlled catalysis in which the incorporation of a nucleotide is slowed, paused, or temporarily inhibited in order to detect a signal and call a correct base. The complex formed during the complexation condition may include a polymerase, template polynucleotide, complementary polynucleotide, and one of a plurality of free nucleotides that is complementary to the 3′-most nucleotide of the 5′ end of the template polynucleotide overhanging the complementary polynucleotide.

The chemical incorporation of a nucleotide may be slowed, paused, or temporarily inhibited in order to detect the signal and call the correct base during a complexation condition. In some examples, the complexation condition includes a non-catalytic metal cation. Examples of non-catalytic metal cations include, but are not limited to one or more of Ca²⁺, Zn²⁺, Co²⁺, Ni²⁺, Eu²⁺, Sr²⁺, Ba²⁺, Fe²⁺, Eu²⁺, and any combination thereof. The concentration of the non-catalytic metal cation present may, in some examples, be less than or equal to about 100 mM. For example, the concentration of the non-catalytic metal may be about 0.1 mM to about 100 mM, or about 1 mM to about 100 mM, or about 5 mM to about 100 mM, or about 5 mM to about 80 mM, or about 10 mM to about 50 mM. In one example, the concentration of the non-catalytic metal cation present during the complexation condition may be less than or equal to about 10 mM, e.g., may be about 0.1 mM to about 10 mM, or about 0.2 mM to about 10 mM, or about 0.5 mM to about 10 mM, or about 1 mM to about 10 mM, or about 2 mM to about 8 mM, or about 2 mM to about 5 mM, or about 5 mM to about 8 mM.

In some examples, the complexation condition includes a chelating agent. Examples of chelating agent include, but are not limited to, ethylene glycol-bis(β-aminoethyl ether)-N,N,N′,N′-tetraacetic acid (EGTA), nitriloacetic acid, tetrasodium iminodisuccinate, ethylene glycol tetraacetic acid, polyaspartic acid, ethylenediamine-N,N′-disuccinic acid (EDDS), methylglycindiacetic acid (MGDA), and any combination thereof.

In some examples, the complexation condition further includes an inhibitor selected from the group consisting of a non-competitive inhibitor, a competitive inhibitor, and a combination thereof.

In some examples, the complexation condition includes a non-competitive inhibitor. The non-competitive inhibitor may be, for example, one or more of an aminoglycoside, a pyrophosphate analog, a melanin, a phosphonoacetate, a hypophosphate, and a rifamycin. Examples of non-competitive inhibitors that may be useful in the complexation condition of the present disclosure include but are not limited to Abacavir hemisulfate (reverse transcriptase inhibitor; antiretroviral); Actinomycin D (inhibits RNA polymerase); Acyclovir (inhibits viral DNA polymerase; antiherpetic agent); AM-TS23 (DNA polymerase λ, and β inhibitor); α-Amanitin (inhibits RNA polymerase II); Aphidicolin (DNA polymerase α, δ and ε inhibitor); Azidothymidine (selective reverse transcriptase inhibitor; antiretroviral); BMH 21 (RNA polymerase 1 inhibitor; also p53 pathway activator); BMS 986094 (prodrug of HCV RNA polymerase inhibitor 2′-C-methyl guanosine triphosphate; potent HCV replication inhibitor); Delavirdine mesylate (non-nucleoside reverse transcriptase inhibitor); Entecavir (potent and selective hepatitis B virus inhibitor); Mithramycin A (inhibitor of DNA and RNA polymerase); Tenofovir (reverse transcriptase inhibitor); and Thiolutin (bacterial RNA polymerase inhibitor).

In some examples, the complexation condition includes a competitive inhibitor. Examples of competitive inhibitors that may be useful in the complexation condition of the present disclosure include but are not limited to aphidicolin, beta-D-arabinofuranosyl-CTP, amiloride, dehydroaltenusin, and any combination thereof.

The pH may also be set to facilitate and/or maintain complexation conditions. In some examples, the complexation condition includes a pH that is less than about 6. The pH may be, for example about 0.1 to about 7, or about 1 to about 7, or about 2 to about 7, or about 2 to about 6, or about 2 to about 5, or about 3 to about 7, or about 7 to about 13.9, or about 7 to about 13, or about 7 to about 12, or about 8 to about 12, or about 8 to about 11.

In some examples, the complexation condition includes a solvent additive. Examples of solvent additives that may be useful in the complexation condition of the present disclosure include but are not limited to ethanol, methanol, tetrahydrofuran, dioxane, dimethylamine (DMA), dimethylformamide (DMF), dimethyl sulfoxide (DMSO), D₂O, lithium, L-cysteine, and a combination thereof. In some examples, the complexation condition includes deuterium.

Changes in conditions may facilitate the transition from a complexation condition to a polymerization condition. Alternatively, as noted above, a change in condition need not necessarily be used to transition from a complexation condition to a polymerization condition. A polymerization condition as described herein may promote the formation of a complex that allows for incorporated of a nucleotide onto the 3′ end of the complementary polynucleotide by the polymerase of the complex. The transition from a complexation condition (also referred to herein as non-incorporating condition) to a polymerization condition (also referred to herein as incorporating condition) may be achieved by, for example, switching from non-catalytic to catalytic conditions, so that the DNA polymerase may incorporate a nucleotide to the DNA, thereby causing dissociation of a leaving group which may carry with it a fluorescent dye attached thereto. The polymerization step may be allowed to proceed for a time sufficient to allow incorporation of a nucleotide.

Polymerases in accordance with the present disclosure may include any polymerase that may tolerate incorporation of a phosphate-labeled nucleotide. Examples of polymerases that may be useful, e.g., in examples such as described with reference to FIGS. 1A-1B, 5A-5B, and 6A-6B, include but are not limited to phi29 polymerase, a Klenow fragment, DNA polymerase I, DNA polymerase III, GA-1, PZA, phi15, phi29, Nf, G1, PZE, PRD1, B103, 9oN polymerase, Bst, Bsu, T4, T5, T7, Taq, Vent, RT, pol beta, VMY22, M2Y, and pol gamma, and mutants thereof. Polymerases engineered to have specific properties may also be used. In some examples, the polymerase may be “processive,” meaning the polymerase remains substantially bound to the primer/template complex throughout multiple incorporation events, e.g., throughout the incorporation of up to hundreds, thousands, or tens of thousands of bases, or more. Nonlimiting examples of processive polymerases include phi29, PZA, GA-1, VMY22, M2Y, and Nf.

The polymerization condition may include various concentrations of Mg²⁺ ions, Mn²⁺ ions, or a combination thereof. For example, the concentration of the Mg′ ions may be about 0.1 mM to about 100 mM, or about 0.1 mM to about 50 mM, or about 0.2 mM to about 50 mM, or about 0.5 mM to about 20 mM, or about 0.1 mM to about 10 mM, or about 1 mM to about 10 mM. Similarly, the concentration of the Mn²⁺ ions may be about 0.1 mM to about 100 mM, or about 0.1 mM to about 50 mM, or about 0.2 mM to about 50 mM, or about 0.5 mM to about 20 mM, or about 0.1 mM to about 10 mM, or about 1 mM to about 10 mM.

### Working Example

The following example is intended to be purely illustrative, and not limiting.

FIGS. 12A-12B schematically illustrate plots of example fluorescence measurements. More specifically, a phi29 processive polymerase was noncovalently coupled to a first oligonucleotide coupled to a quencher, and covalently coupled to a second oligonucleotide coupled to a fluorophore in a manner similar to that described with reference to FIGS. 1A-1B, but in solution rather than coupled to a substrate. In a manner such as described with reference to FIGS. 2A-2B, the first and second oligonucleotides were complementary to each other. The resulting composition was contacted with nucleotides coupled to labels each including a third oligonucleotide that was complementary to the first oligonucleotide, and a target polynucleotide to be sequenced, plus a primer, under non-catalytic conditions. From this example, it may be understood that the action of the polymerase upon the nucleotide in its active site generates a higher local concentration of the third oligonucleotide (“trapping” the third oligonucleotide), which causes preferential association between the second and third oligonucleotides and inhibits rebinding of the first oligonucleotide to which the quencher is coupled. For nucleotides for which the third oligonucleotide is complementary to the second oligonucleotide (“correct”), an increase of fluorescence 1210 was observed, as illustrated in FIG. 12A, that is indicative of disassociation between the quencher and the fluorophore, resulting from displacement of the second oligonucleotide by the third oligonucleotide. In comparison, for nucleotides for which the third oligonucleotide was not complementary to the second oligonucleotide (“incorrect”), a decrease of fluorescence 1220 was observed, as illustrated in FIG. 12A, that is indicative that the quencher and the fluorophore substantially did not disassociate because there was substantially no displacement of the second oligonucleotide by the third oligonucleotide. Upon addition of Mn²⁺ in the same pot, a decrease in fluorescence 1210′ was observed that is indicative of disassociation of the third (“correct”) oligonucleotide and quencher re-association, as illustrated in FIG. 12B, with continued low fluorescence 1220′ for the third (“incorrect”) oligonucleotide. The complete cycle was observed in a single pot and is expected to be repeatable so as to allow for identifying nucleotides using fluorescence during sequencing of the entire target polynucleotide.

Additional Notes

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method comprising:
adding, using a polymerase coupled to a substrate, nucleotides in a solution to a first polynucleotide using at least a sequence of a second polynucleotide;
separating, using labels respectively coupled to the nucleotides, quenchers from respective fluorophores; and
detecting a sequence in which the polymerase adds the nucleotides to the first polynucleotide using at least fluorescence from the respective fluorophores.

2. The method of claim 1, wherein the quenchers are coupled to the substrate.

3. The method of claim 1, wherein the fluorophores are coupled to the substrate.

4. The method of claim 3, wherein the fluorophores are coupled to respective quenchers via non-covalent associations.

5. The method of claim 4, wherein the separating comprises the labels disrupting the non-covalent associations.

6. The method of claim 4, wherein each of the fluorophores is coupled to a respective first oligonucleotide, each of the quenchers is coupled to a respective second oligonucleotide, and the non-covalent associations comprise hybridizations between the first oligonucleotides and the second oligonucleotides.

7. The method of claim 6, wherein each of the labels comprises a respective third oligonucleotide.

8. The method of claim 7, wherein the third oligonucleotides disrupt the non-covalent associations by hybridizing to the first oligonucleotides.

9. The method of claim 7, wherein the third oligonucleotides disrupt the non-covalent associations by hybridizing to the second oligonucleotides.

10. The method of claim 4, wherein each of the fluorophores and respective quencher is coupled to a respective hairpin oligonucleotide having first and second stem sequences and a loop sequence, and the non-covalent associations comprise hybridizations between the first and second stem sequences.

11. The method of claim 10, wherein each of the labels comprises a respective oligonucleotide.

12. The method of claim 11, wherein the oligonucleotides disrupt the non-covalent associations by hybridizing to the loop sequences.

13. The method of claim 1, wherein after the polymerase adds respective nucleotides, the non-covalent associations re-form between the fluorophores and respective quenchers.

14. The method of claim 1, wherein the quenchers are in the solution.

15. The method of claim 14, wherein the fluorophores are coupled to respective labels.

16. The method of claim 15, wherein the fluorophores are coupled to respective quenchers via non-covalent associations.

17. The method of claim 16, wherein an element coupled to the surface disrupts the non-covalent associations.

18. The method of claim 17, wherein each of the labels is coupled to a respective first oligonucleotide to which a respective fluorophore is coupled, each of the quenchers is coupled to a respective second oligonucleotide, and the non-covalent associations comprise hybridizations between the first oligonucleotides and the second oligonucleotides.

19. The method of claim 18, wherein the element coupled to the surface comprises a third oligonucleotide.

20. The method of claim 19, wherein the third oligonucleotide disrupts the non-covalent associations by hybridizing to the first oligonucleotide.

21. The method of claim 19, wherein the third oligonucleotide disrupts the non-covalent associations by hybridizing to the second oligonucleotides.

22. The method of claim 16, wherein each of the fluorophores and respective quencher is coupled to a respective hairpin oligonucleotide having first and second stem sequences and a loop sequence, and the non-covalent associations comprise hybridizations between the first and second stem sequences.

23. The method of claim 22, wherein the element coupled to the surface comprises an oligonucleotide.

24. The method of claim 23, wherein the oligonucleotide disrupts the non-covalent associations by hybridizing to the loop sequence.

25. The method of claim 14, wherein after the polymerase adds respective nucleotides, new non-covalent associations form between the fluorophores and respective quenchers in the solution.

26. A composition comprising:
a substrate;
a polymerase coupled to the substrate;
a plurality of quenchers coupled to the substrate; and
a plurality of fluorophores, each fluorophore being non-covalently associated with at least one of the quenchers.

27. The composition of claim 26, further comprising:
a solution comprising nucleotides having labels coupled thereto; and
optical detection circuitry, wherein:
the polymerase is to add the nucleotides to a first polynucleotide using at least a sequence of a second polynucleotide,
the labels disrupt the non-covalent associations between the fluorophores and the quenchers, and
the optical detection circuitry is to detect fluorescence from the fluorophores resulting from the disruption in the non-covalent associations.

28. The composition of claim 26, wherein the fluorophores are coupled to the substrate.

29. The composition of claim 27, wherein each of the fluorophores is coupled to a respective first oligonucleotide, each of the quenchers is coupled to a respective second oligonucleotide, and the non-covalent associations comprise hybridizations between the first oligonucleotides and the second oligonucleotides.

30. The composition of claim 29, wherein each of the labels comprises a respective third oligonucleotide.

31. The composition of claim 30, wherein the third oligonucleotide disrupts the non-covalent associations by hybridizing to the first oligonucleotide.

32. The composition of claim 30, wherein the third oligonucleotide disrupts the non-covalent associations by hybridizing to the second oligonucleotide.

33. The composition of claim 27, wherein each of the fluorophores and respective quencher is coupled to a respective hairpin oligonucleotide having first and second stem sequences and a loop sequence, and the non-covalent associations comprise hybridizations between the first and second stem sequences.

34. The composition of claim 33, wherein each of the labels comprises a respective oligonucleotide.

35. The composition of claim 34, wherein the oligonucleotides disrupt the non-covalent associations by hybridizing to the loop sequences.

36. The composition of claim 27, wherein after the polymerase adds respective nucleotides, the non-covalent associations re-form between the fluorophores and respective quenchers.

37. A composition comprising:
a substrate;
a polymerase coupled to the substrate; and
a solution comprising (i) quenchers and (ii) nucleotides coupled to labels, each label being coupled to a fluorophore, each fluorophore being non-covalently associated with at least one of the quenchers; and
an element coupled to the substrate to disrupt the non-covalent associations between the fluorophores and the quenchers.

38. The composition of claim 37, further comprising optical detection circuitry, wherein:
the polymerase is to add the nucleotides to a first polynucleotide using at least a sequence of a second polynucleotide, and
the optical detection circuitry is to detect fluorescence from the fluorophores resulting from the disruption in the non-covalent associations.

39. The composition of claim 37, wherein each of the labels is coupled to a respective first oligonucleotide to which a respective fluorophore is coupled, each of the quenchers is coupled to a respective second oligonucleotide, and the non-covalent associations comprise hybridizations between the first oligonucleotides and the second oligonucleotides.

40. The composition of claim 39, wherein the element coupled to the surface comprises a third oligonucleotide.

41. The composition of claim 40, wherein the third oligonucleotide is to disrupt the non-covalent associations by hybridizing to the first oligonucleotides.

42. The composition of claim 40, wherein the third oligonucleotide is to disrupt the non-covalent associations by hybridizing to the second oligonucleotides.

43. The composition of claim 37, wherein each of the fluorophores and respective quencher is coupled to a respective hairpin oligonucleotide having first and second stem sequences and a loop sequence, and the non-covalent associations comprise hybridizations between the first and second stem sequences.

44. The composition of claim 43, wherein the element coupled to the surface comprises an oligonucleotide.

45. The composition of claim 44, wherein the oligonucleotide is to disrupt the non-covalent associations by hybridizing to the loop sequences.

46. The composition of claim 37, wherein after the polymerase adds respective nucleotides, new non-covalent associations form between the fluorophores and respective quenchers in the solution.

47-71. (canceled)
